(19) United States
(12) Patent Application Publication
(10) Pub. No.: US 2022/0296079 A1
(43) Pub. Date: Sep. 22, 2022

(54) CONTROL DEVICE, METHOD FOR CHANGING RIGIDITY OF INSERTION PORTION OF ENDOSCOPE, AND RECORDING MEDIUM

(71) Applicant: OLYMPUS CORPORATION, Tokyo (JP)

(72) Inventor: Takeshi TAKAHASHI, Tokyo (JP)

(73) Assignee: OLYMPUS CORPORATION, Tokyo (JP)

(21) Appl. No.: 17/834,130

(22) Filed: Jun. 7, 2022

Related U.S. Application Data
(63) Continuation of application No. PCT/JP2019/048121, filed on Dec. 9, 2019.

(51) Int. Cl. A61B 1/005; A61B 1/00

(52) U.S. Cl. CPC A61B 1/0057; A61B 1/00071; A61B 1/0055; A61B 1/009

(57) ABSTRACT

A control device includes a processor including at least one hardware unit, the control device controlling an endoscope having a channel through which a treatment instrument is inserted, the endoscope including an insertion portion configured to be inserted into a subject and a rigidity variable unit provided in the insertion portion and configured to be capable of partially changing rigidity of the insertion portion. The processor detects formation of a bent portion in the insertion portion based on a detection result of a shape of the insertion portion, and in a case where the processor detects the formation of the bent portion in the insertion portion, the processor performs control of increasing rigidity of the rigidity variable unit located on a proximal end side in the bent portion.

### CROSS REFERENCE TO RELATED APPLICATION

This application is a continuation application of PCT/JP2019/048121 filed on Dec. 9, 2019, the entire contents of which are incorporated herein by this reference.

### BACKGROUND OF THE INVENTION

### 1. Field of the Invention

The present invention relates to a control device, to a method for changing rigidity of an insertion portion of an endoscope, and to a recording medium. The present invention particularly relates to a control device including an endoscope having an insertion portion in which a rigidity variable unit is provided, to a method for changing rigidity of an insertion portion of an endoscope, and to a recording medium.

### 2. Description of the Related Art

Conventionally, there has been a widely known technique of inserting an elongated insertion portion having flexibility into a deep part of a subject. For example, in a medical field, there has been a widely known technique of observing an object in the subject or of performing various therapeutic treatments by using an endoscope having the elongated insertion portion.

In general, the insertion portion of the endoscope of this kind is formed such that a distal end rigid portion, a bending portion, and a flexible portion (flexible tube portion) are arranged in this order from a distal end of the insertion portion. When an operator inserts the insertion portion of the endoscope into a body cavity, being a subject, the operator pushes the insertion portion into the body cavity while grasping the flexible portion (flexible tube portion), and causes the bending portion to bend in a desired direction by operating an operation knob provided on an operation portion of the endoscope.

As an example of performing various therapeutic treatments using the endoscope having the insertion portion of this kind, there is a known example where a predetermined treatment instrument is inserted through a channel formed in the endoscope and treatment is performed using the treatment instrument. For example, in a case of performing therapeutic treatment, such as endoscopic submucosal dissection (ESD), there is a known example where a lesion is dissected by using a treatment instrument, such as a so-called IT knife, projecting out from a channel opening formed in the endoscope.

In such treatment, after the IT knife is caused to approach the target lesion, dissection is performed with the IT knife by taking full advantage of operations of a distal end portion of the insertion portion, such as a bending operation, a twisting operation, and an advancing and retracting operation. In performing dissection work with the IT knife along the lesion, in order to accurately perform treatment, it is necessary to surely transmit operations (bending, twisting, and advancing and retracting) of the operation portion of the endoscope (also being a grasping portion of the endoscope) to the distal end portion.

In a case where the elongated insertion portion is inserted into the body cavity of the subject, the insertion portion may be brought into a bending state in the body cavity. There may be a situation where therapeutic treatment using the IT knife or other instruments is performed as described above with the insertion portion being in the bending state as described above.

In WO2016/151846, the applicant of the present invention proposes an endoscope system where flexural rigidity (hardness) can be changed according to an insertion state (bending state) of an insertion portion of an endoscope. However, an object of this endoscope system is to improve ease of insertion of a distal end portion of the insertion portion by increasing rigidity of a bending portion of the insertion portion on a distal end side. In contrast, in order to improve stability of a hand-side operation in performing treatment by inserting a treatment instrument through a channel of the insertion portion, there has been a demand for a technique that increases rigidity of the bending portion on a proximal end side.

### SUMMARY OF THE INVENTION

One aspect of the present invention is directed to a control device that controls an endoscope having a channel through which a treatment instrument is inserted, the endoscope including an insertion portion configured to be inserted into a subject from a distal end side of the insertion portion and one rigidity variable unit or a plurality of rigidity variable units provided in the insertion portion and configured to be capable of partially changing rigidity of the insertion portion, the control device including a processor including at least one hardware unit. The processor detects formation of a bent portion in the insertion portion based on a detection result of a shape of the insertion portion, and in a case where the processor detects the formation of the bent portion in the insertion portion, the processor performs control of increasing rigidity of the rigidity variable unit located on a proximal end side in the bent portion.

Another aspect of the present invention is directed to a method for changing rigidity of an insertion portion of an endoscope having a channel through which a treatment instrument is inserted, the endoscope including an insertion portion configured to be inserted into a subject from a distal end side of the insertion portion and one rigidity variable unit or a plurality of rigidity variable units provided in the insertion portion and configured to be capable of partially changing rigidity of the insertion portion, the method including: detecting formation of a bent portion in the insertion portion; and increasing rigidity of the rigidity variable unit located on a proximal end side in the bent portion in a case where the formation of the bent portion in the insertion portion is detected.

Still another aspect of the present invention is directed to a non-transitory recording medium in which a program is recorded, the program causing a computer to execute processing of controlling rigidity of an insertion portion of an endoscope having a channel through which a treatment instrument is inserted, the endoscope including an insertion portion configured to be inserted into a subject from a distal end side of the insertion portion and one rigidity variable unit or a plurality of rigidity variable units provided in the insertion portion and configured to be capable of partially changing the rigidity of the insertion portion, wherein the non-transitory recording medium causes the computer to execute processing of detecting formation of a bent portion in the insertion portion based on a detection result of a shape of the insertion portion, and processing of increasing rigidity of the rigidity variable unit located on a proximal end side in the bent portion in a case where the formation of the bent portion in the insertion portion is detected.

### BRIEF DESCRIPTION OF THE DRAWINGS

FIG. 1 is a block diagram showing a configuration of an endoscope system according to a first embodiment of the present invention;

FIG. 2 is a block diagram showing an electrical configuration of the endoscope system according to the first embodiment;

FIG. 3 is an explanatory view showing control of a rigidity variable unit when a bent portion occurs in an insertion portion of an endoscope of the endoscope system according to the first embodiment in a state where the insertion portion is inserted into the body cavity of a subject;

FIG. 4 is an enlarged view of a main part showing a state where the bent portion of the insertion portion of the endoscope of the endoscope system according to the first embodiment is pressed against an inner wall of a body;

FIG. 5 is a graph for describing a situation where the bent portion occurs in the insertion portion of the endoscope of the endoscope system according to the first embodiment;

FIG. 6 is a block diagram showing an electrical configuration of an endoscope system according to a second embodiment of the present invention;

FIG. 7 is a block diagram showing a configuration of an endoscope system according to a third embodiment of the present invention;

FIG. 8 is a block diagram showing an electrical configuration of the endoscope system according to the third embodiment;

FIG. 9 is a graph for describing a situation where a bent portion occurs in an insertion portion of an endoscope of the endoscope system according to the third embodiment;

FIG. 10 is a block diagram showing a configuration of an endoscope system according to a fourth embodiment of the present invention;

FIG. 11 is a block diagram showing an electrical configuration of the endoscope system according to the fourth embodiment;

FIG. 12 is an explanatory view showing control of rigidity variable units when a bent portion occurs in an insertion portion of an endoscope of the endoscope system according to the fourth embodiment in a state where the insertion portion is inserted into the body cavity of a subject;

FIG. 13 is an enlarged view of a main part showing a state where the bent portion of the insertion portion of the endoscope of the endoscope system according to the fourth embodiment is pressed against the inner wall of the body;

FIG. 14 is a block diagram showing a configuration of an endoscope system according to a fifth embodiment of the present invention;

FIG. 15 is a block diagram showing an electrical configuration of the endoscope system according to the fifth embodiment;

FIG. 16 is an enlarged view of a main part showing a treatment instrument insertion sensor of the endoscope system according to the fifth embodiment;

FIG. 17 is an enlarged perspective view of a main part showing the treatment instrument insertion sensor of the endoscope system according to the fifth embodiment;

FIG. 18 is a block diagram showing an electrical configuration of an endoscope system according to a sixth embodiment of the present invention;

FIG. 19 is a view for describing determination made by a treatment determination unit of the endoscope system according to the sixth embodiment after the treatment determination unit receives image processing information;

FIG. 20 is a view for describing a manner of operation of an insertion portion stability calculation unit of an endoscope system according to a seventh embodiment of the present invention;

FIG. 21 is a view for describing the manner of operation of the insertion portion stability calculation unit of the endoscope system according to the seventh embodiment;

FIG. 22 is a view for describing the manner of operation of the insertion portion stability calculation unit of the endoscope system according to the seventh embodiment;

FIG. 23 is a view showing another configuration example applicable for the rigidity variable unit of the endoscope system according to any one of the first to seventh embodiments;

FIG. 24 is a view showing another configuration example applicable for the rigidity variable unit of the endoscope system according to any one of the first to seventh embodiments;

FIG. 25 is a view showing another configuration example applicable for the rigidity variable unit of the endoscope system according to any one of the first to seventh embodiments;

FIG. 26 is a view showing another configuration example applicable for the rigidity variable unit of the endoscope system according to any one of the first to seventh embodiments;

FIG. 27 is a view showing another configuration example applicable for the rigidity variable unit of the endoscope system according to any one of the first to seventh embodiments; and

FIG. 28 is a view showing another configuration example applicable for the rigidity variable unit of the endoscope system according to any one of the first to seventh embodiments.

### DETAILED DESCRIPTION OF THE PREFERRED EMBODIMENTS

Hereinafter, embodiments of the present invention will be described with reference to drawings.

### First Embodiment

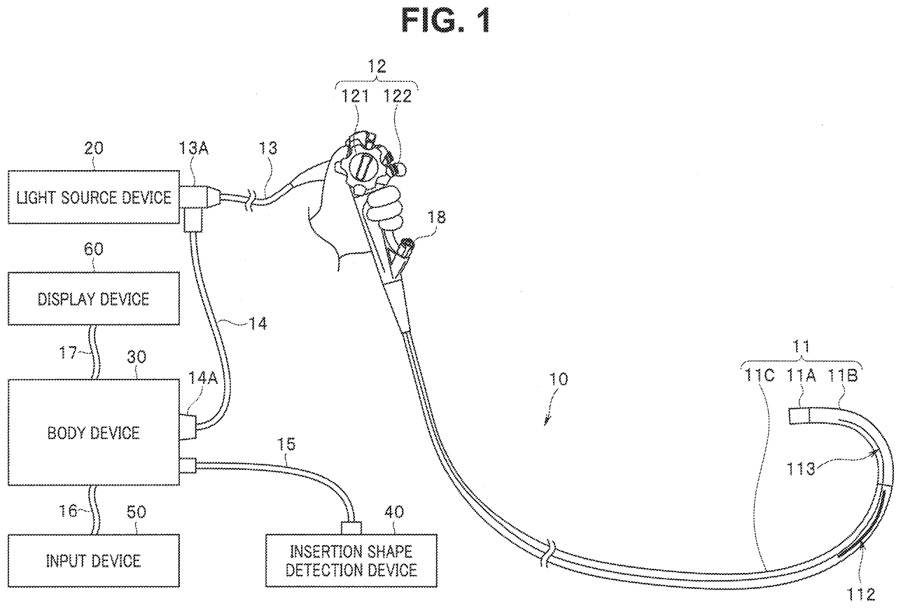

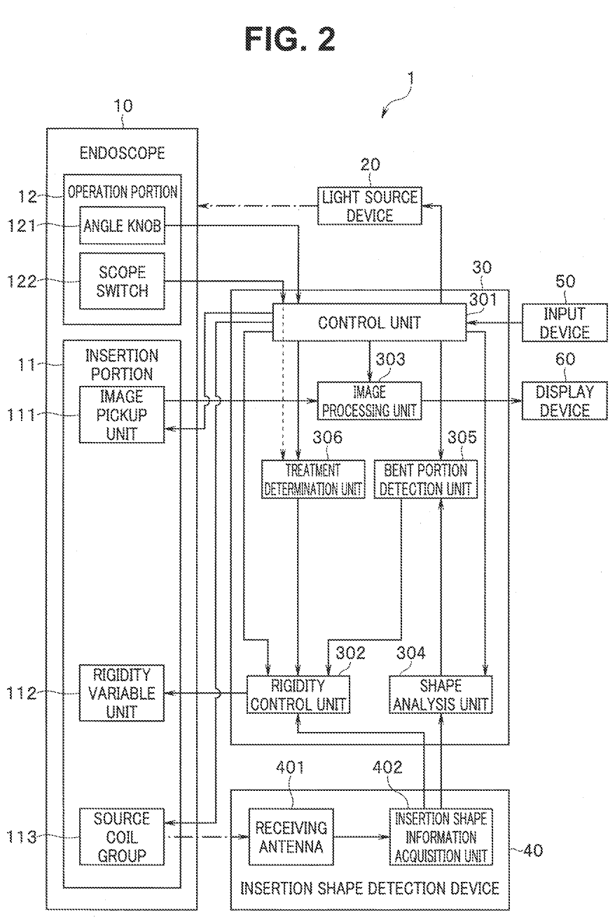

FIG. 1 is a block diagram showing a configuration of an endoscope system according to a first embodiment of the present invention. FIG. 2 is a block diagram showing an electrical configuration of the endoscope system of the first embodiment.

It is assumed that an endoscope system 1 of the first embodiment is an endoscope system that includes a so-called large intestine endoscope being inserted into the intestinal tract of a subject.

As shown in FIG. 1, the endoscope system 1 according to the first embodiment is configured to include, for example, an endoscope 10, a light source device 20, a body device 30, an insertion shape detection device 40, an input device 50, and a display device 60.

The endoscope 10 is configured to include an insertion portion 11, an operation portion 12, a universal cord 13, and a channel opening portion 18. The insertion portion 11 is inserted into the subject. The operation portion 12 is provided on a proximal end side of the insertion portion 11. The universal cord 13 extends from the operation portion 12. The channel opening portion 18 allows insertion of a treatment instrument. The endoscope 10 is configured to be removably connected to the light source device 20 via a scope connector 13A, which is provided to an end portion of the universal cord 13.

The endoscope 10 is also configured to be removably connected to the body device 30 via an electrical connector 14A, which is provided to an end portion of an electric cable 14 extending from the scope connector 13A. A light guide (not shown in the drawing) is provided in the insertion portion 11, the operation portion 12, and the universal cord 13 to transmit illumination light supplied from the light source device 20.

The insertion portion 11 is configured to have flexibility and an elongated shape. The insertion portion 11 is formed by providing a rigid distal end portion 11A, a bending portion 11B, and a long flexible tube portion 11C in this order from a distal end side of the insertion portion 11. The bending portion 11B is formed to be bendable. The flexible tube portion 11C has flexibility.

A source coil group 113 (see FIG. 2) is provided in the distal end portion 11A, the bending portion 11B, and the flexible tube portion 11C. In the source coil group 113, a plurality of source coils are arranged at predetermined intervals in a longitudinal direction of the insertion portion 11, the plurality of source coils generating magnetic fields in response to a coil drive signal supplied from the body device 30. The source coil group 113 forms a so-called endoscope insertion shape detection device (UPD).

The distal end portion 11A is provided with an illumination window (not shown in the drawing) to emit illumination light to an object, the illumination light being transmitted through the light guide provided in the insertion portion 11. The distal end portion 11A is also provided with an image pickup unit 111 (see FIG. 2).

The image pickup unit 111 is configured to perform an action in response to an image pickup control signal supplied from the body device 30, and is configured to pick up an image of the object illuminated by the illumination light emitted through the illumination window and to then output an image pickup signal. The image pickup unit 111 is configured to include an image sensor, such as a CMOS image sensor or a CCD image sensor.

The bending portion 11B is configured to be able to bend according to an operation of an angle knob 121 provided to the operation portion 12.

Although described later in detail, a rigidity variable unit 112 is provided in the longitudinal direction of the insertion portion 11 in a rigidity variable range that corresponds to a predetermined range from a proximal end portion of the bending portion 11B to a distal end portion of the flexible tube portion 11C. The rigidity variable unit 112 is configured to be able to change flexural rigidity in the rigidity variable range according to control from the body device 30. A specific configuration and the like of the rigidity variable unit 112 will be described later in detail.

Hereinafter, for the sake of convenience of description, “flexural rigidity” is simply referred to as “rigidity” when appropriate. Further, in the present embodiment, it is sufficient that the above-mentioned rigidity variable range is provided to at least a portion of the insertion portion 11.

The operation portion 12 is configured to have a shape that allows a user to grasp and operate the operation portion 12. Further, the operation portion 12 is provided with the angle knob 121 that is configured to allow the user to perform an operation of bending the bending portion 11B in four directions (UDLR) of an upward direction, a downward direction, a leftward direction, or a rightward direction intersecting with a longitudinal axis of the insertion portion 11. The operation portion 12 is also provided with one or more scope switches 122 each of which can perform an instruction corresponding to an input operation performed by the user.

The light source device 20 is configured to include, for example, one or more LEDs or one or more lamps as a light source. The light source device 20 is also configured to generate illumination light for illuminating the inside of the subject into which the insertion portion 11 is inserted, and configured to be able to supply the illumination light to the endoscope 10. The light source device 20 is also configured to be able to change an amount of illumination light according to a system control signal supplied from the body device 30.

The body device 30 is configured to be removably connected to the insertion shape detection device 40 via a cable 15. The body device 30 is also configured to be removably connected to the input device 50 via a cable 16. Further, the body device 30 is configured to be removably connected to the display device 60 via a cable 17.

The body device 30 is also configured to perform an action corresponding to an instruction from the input device 50 and instructions from the scope switch 122 and the angle knob 121. The body device 30 is also configured to form an endoscope image based on an image pickup signal outputted from the endoscope 10, and configured to perform an action of causing the display device 60 to display the formed endoscope image. Further, the body device 30 is configured to generate and output various control signals for controlling actions of the endoscope 10 and the light source device 20.

In the present embodiment, the body device 30 is configured such that a rigidity control unit 302, which will be described later, controls a driving state of the rigidity variable unit 112 based on insertion shape information (described later) or the like outputted from the insertion shape detection device 40 (see FIG. 2).

### Configuration of Rigidity Variable Unit 112 in Present Embodiment

A configuration of the rigidity variable unit 112 adopted in the first embodiment will be described.

In the first embodiment, the rigidity variable unit 112 is formed as an actuator including, for example, a coil heater and a shape memory member not shown in the drawing, and is provided in the longitudinal direction of the insertion portion 11 within the predetermined range (preferably equal to or less than 150 mm) from the proximal end portion of the bending portion 11B to the distal end portion of the flexible tube portion 11C. In the present embodiment, the rigidity variable unit 112 is formed as a rod-like actuator where cross sections orthogonal to the longitudinal direction have the same shape.

The coil heater is formed by winding a winding wire having high thermal conductivity, such as a nichrome wire, into a cylindrical shape. Therefore, the rigidity variable unit 112 is configured to generate heat according to control from the rigidity control unit 302.

In contrast, the shape memory member of the rigidity variable unit 112 is formed as an elongated member including a shape memory alloy, such as nickel titanium, and is disposed in a state of being inserted through an inner space of the coil heater. The shape memory member is configured to be able to change elasticity of the shape memory member according to heat generated from the coil heater.

Specifically, the shape memory member is configured as follows. When the shape memory member is heated to a temperature equal to or above a temperature TN higher than at least room temperature by heat generated from the coil heater, for example, the shape memory member is brought into a high elasticity state having a restoring force that restores the shape memory member to a linear shape corresponding to a shape memorized in advance.

The shape memory member is configured such that, for example, when the shape memory member is not heated to a temperature equal to or above the temperature TN due to factors such as heat not being generated from the coil heater, the shape memory member is brought into a low elasticity state having no restoring force that restores the shape memory member to the linear shape corresponding to the shape memorized in advance.

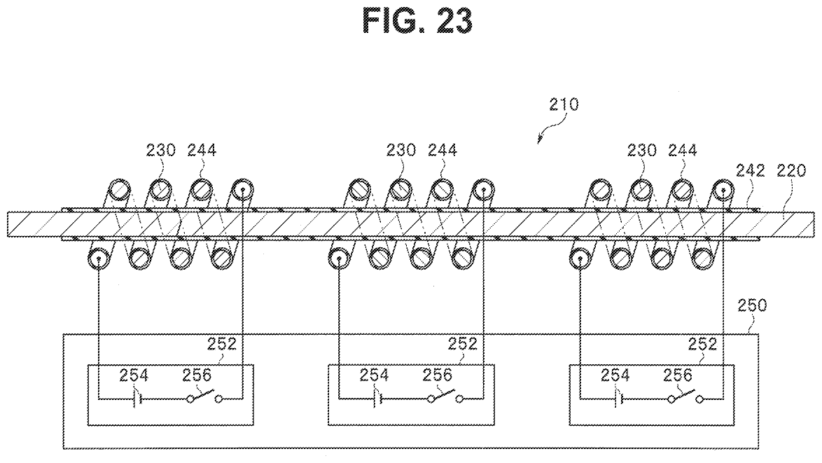

In the first embodiment, the rigidity variable unit 112 is formed as the actuator including the coil heater and the shape memory member. However, the configuration of the rigidity variable unit 112 is not limited to such a configuration. For example, as shown in FIG. 23 to FIG. 28, various configurations may be adopted. Other configuration examples of the rigidity variable unit 112 will be described later in detail.

Returning to FIG. 2, the insertion shape detection device 40 forms a so-called endoscope insertion shape detection device (UPD), and is configured to detect magnetic fields generated from the source coil group 113 provided to the insertion portion 11, and is configured to acquire respective positions of the plurality of source coils, included in the source coil group 113, based on magnitudes of the detected magnetic fields.

The insertion shape detection device 40 is also configured to calculate an insertion shape of the insertion portion 11 based on the respective positions of the plurality of source coils acquired as described above, and configured to generate insertion shape information indicating the calculated insertion shape and to then output the insertion shape information to the body device 30. The insertion shape detection device 40 will be described later in detail.

The input device 50 is configured to include one or more input interfaces operated by the user, such as a mouse, a keyboard, and a touch panel. The input device 50 is also configured to be able to output, to the body device 30, an instruction corresponding to an operation performed by the user.

The display device 60 is configured to include a liquid crystal monitor, for example. The display device 60 is also configured to be able to display an endoscope image or the like, outputted from the body device 30, on a screen.

### <Configuration of Insertion Shape Detection Device 40>

As shown in FIG. 2, the insertion shape detection device 40 is configured to include a receiving antenna 401 and an insertion shape information acquisition unit 402.

The receiving antenna 401 is configured to include, for example, a plurality of coils that three-dimensionally detect magnetic fields generated from the plurality of respective source coils included in the source coil group 113. The receiving antenna 401 is also configured to detect the magnetic fields generated from the plurality of respective source coils included in the source coil group 113, and configured to generate a magnetic field detection signal corresponding to the magnitudes of the detected magnetic fields and to then output the magnetic field detection signal to the insertion shape information acquisition unit 402.

The insertion shape information acquisition unit 402 is configured to acquire respective positions of the plurality of source coils included in the source coil group 113 based on the magnetic field detection signal outputted from the receiving antenna 401.

The insertion shape information acquisition unit 402 is also configured to calculate an insertion shape of the insertion portion 11 based on the respective positions of the plurality of source coils acquired as described above, and configured to generate insertion shape information indicating the calculated insertion shape and to then output the insertion shape information to the rigidity control unit 302 and a shape analysis unit 304 of the body device 30.

Specifically, as the respective positions of the plurality of source coils included in the source coil group 113, the insertion shape information acquisition unit 402 acquires, for example, a plurality of three-dimensional coordinate values in a space coordinate system where a predetermined position (anus or the like) of the subject, into which the insertion portion 11 is inserted, is virtually set as an origin or a reference point.

As processing for calculating the insertion shape of the insertion portion 11, the insertion shape information acquisition unit 402 performs, for example, interpolation processing for interpolating the plurality of three-dimensional coordinate values acquired as described above.

In the present embodiment, each unit of the insertion shape detection device 40 may be an electronic circuit, or may be a circuit block of an integrated circuit, such as an FPGA (field programmable gate array). In the present embodiment, for example, the insertion shape detection device 40 may be configured to include one or more processors (CPUs or the like).

### <Internal Configuration of the Body Device 30>

Next, an internal configuration of the body device 30 in the present embodiment will be described with reference to FIG. 2.

FIG. 2 is a block diagram showing the electrical configuration of the endoscope system according to the first embodiment.

In the present embodiment, as shown in FIG. 2, the body device 30 is configured to include a control unit 301, the rigidity control unit 302, an image processing unit 303, the shape analysis unit 304, a bent portion detection unit 305, and a treatment determination unit 306.

The control unit 301 is configured to generate and output an image pickup control signal that controls an image pickup action of the image pickup unit 111. The control unit 301 is also configured to generate and output a coil drive signal that drives the respective source coils included in the source coil group 113.

The control unit 301 is also configured to generate a system control signal that causes an action to be performed according to an instruction from the input device 50 and instructions from the scope switch 122 and the angle knob 121, and to then output the generated system control signal to a circuit unit, such as the image processing unit 303, in addition to the light source device 20.

Although described later in detail, the control unit 301 controls actions of the rigidity control unit 302, the shape analysis unit 304, the bent portion detection unit 305, and the treatment determination unit 306.

The image processing unit 303 is configured to form an endoscope image by performing predetermined processing on the image pickup signal, outputted from the endoscope 10, in response to the system control signal outputted from the control unit 301, and to then output the formed endoscope image to the display device 60.

### <Shape Analysis Unit 304>

Under control from the control unit 301, the shape analysis unit 304 acquires insertion shape information from the insertion shape information acquisition unit 402 of the insertion shape detection device 40 and, based on the shape information, analyzes a shape of the insertion portion 11 inserted into the body cavity of the subject. Specifically, the shape analysis unit 304 calculates a radius of curvature of the bending insertion portion 11, and then sends the calculation result to the bent portion detection unit 305 provided downstream of the shape analysis unit 304. In the present embodiment, the shape analysis unit 304 calculates the radius of curvature of the insertion portion 11. However, the configuration is not limited to such a configuration. The shape analysis unit 304 may calculate a curvature of the insertion portion 11.

### <Bent Portion Detection Unit 305>

Under control from the control unit 301, the bent portion detection unit 305 detects, based on a value of the radius of curvature of the insertion portion 11 calculated by the shape analysis unit 304, whether a bent portion occurs in the insertion portion 11. Specifically, when the value of the radius of curvature of the insertion portion 11 calculated by the shape analysis unit 304 is equal to or less than a predetermined value, the bent portion detection unit 305 determines that the “bent portion” is formed at a portion of the bending portion of the insertion portion 11, and outputs the determination result to the rigidity control unit 302 provided downstream of the bent portion detection unit 305.

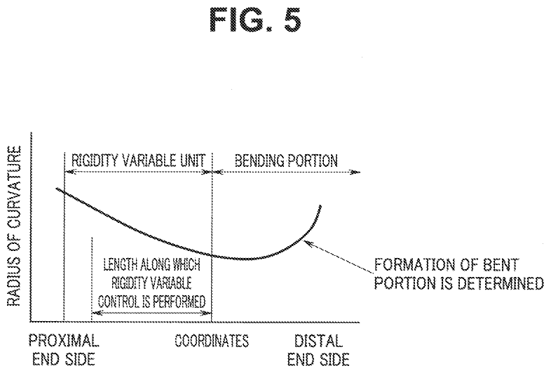

When the bent portion detection unit 305 determines that the “bent portion” is formed in the insertion portion 11, the bent portion detection unit 305 calculates a length along which rigidity variable control of the rigidity variable unit 112 is performed (see FIG. 5). Specifically, according to a bent state of the “bent portion”, that is, according to a size of the radius of curvature of the bent portion and a position where the bent portion occurs, for example, it is necessary to control a corresponding length along which rigidity variable control of the rigidity variable unit 112 is performed (a length from the proximal end portion of the bending portion). Accordingly, in the present embodiment, the bent portion detection unit 305 determines that the “bent portion” is formed in the insertion portion 11, and the bent portion detection unit 305 calculates the length along which rigidity variable control is performed. For example, to calculate a length along which rigidity variable control is performed, the bent portion detection unit 305 stores in advance a relationship between distribution of a bending rate and lengths along which rigidity variable control is performed.

As described above, when the insertion portion 11 bends, so that the “bent portion” occurs, the bent portion detection unit 305 transmits information about the formation of the “bent portion” to the rigidity control unit 302. When the rigidity control unit 302 receives such information, the rigidity control unit 302 performs control of increasing rigidity of the rigidity variable unit 112 as will be described later. However, if rigidity of the rigidity variable unit 112 is increased when a bending operation of the insertion portion 11 is merely performed, a problem may occur instead.

The present invention has been made under such circumstances, and is characterized in that occurrence of the bent portion in the insertion portion 11 is not the only condition for starting rigidity control, but as will be described later, performing treatment using a treatment instrument by an operator is also a condition for starting rigidity control.

### <Treatment Determination Unit 306>

In contrast, under control from the control unit 301, the treatment determination unit 306 determines whether predetermined treatment is about to be performed by the operator. In the present embodiment, the predetermined treatment indicates treatment performed on the subject by using the treatment instrument in a state where the operator inserts the insertion portion 11 of the endoscope 10 into the body cavity of the subject, and a predetermined treatment instrument 125 is inserted from the channel opening portion 18 of the endoscope 10 toward a treatment instrument insertion channel.

That is to say, in the present embodiment, when the operator first inserts the insertion portion 11 into the body cavity of the subject and then inserts the predetermined treatment instrument 125 from the channel opening portion 18 toward the treatment instrument insertion channel, the treatment determination unit 306 determines whether treatment using the treatment instrument is about to be performed in the body cavity of the subject.

Specifically, in the first embodiment, when the treatment determination unit 306 receives an ON signal from the scope switch 122 of the operation portion 12, the treatment determination unit 306 determines that treatment using the treatment instrument is about to be performed.

### <Rigidity Control Unit 302>

The rigidity control unit 302 is configured to perform, under control from the control unit 301, an action for controlling a driving state of the rigidity variable unit 112 based on insertion shape information outputted from the insertion shape information acquisition unit 402 of the insertion shape detection device 40. As described above, the rigidity variable unit 112 is configured such that rigidity variable control is performed by the rigidity control unit 302.

The rigidity control unit 302 is configured to include a drive circuit, a memory, and a control circuit not shown in the drawing. The drive circuit controls the above-mentioned coil heater of the rigidity variable unit 112 according to control from the control circuit. The memory stores predetermined rigidity control information. For example, the memory stores rigidity control information including information indicating a rigidity variable range of the insertion portion 11 and information indicating thresholds corresponding to predetermined parameters calculated for controlling the rigidity variable unit 112. The control circuit controls the drive circuit based on rigidity control information read from the memory and insertion shape information outputted from the insertion shape information acquisition unit 402.

As described above, by the rigidity control unit 302 of the body device 30, the rigidity variable unit 112 in the first embodiment is caused to first perform an action for sequentially increasing flexural rigidity in the rigidity variable range of the insertion portion 11 in a direction from a center portion toward both end portions in the rigidity variable range, for example.

In contrast, in the first embodiment, under control from the control unit 301, the rigidity control unit 302 controls rigidity of the rigidity variable unit 112 based on signals from the bent portion detection unit 305 and the treatment determination unit 306.

Specifically, in the first embodiment, the rigidity control unit 302 obtains a detection result from the bent portion detection unit 305. When the rigidity control unit 302 determines, based on the detection result, that a predetermined “bent portion” is formed at a portion of the insertion portion 11, the rigidity control unit 302 obtains a detection result from the treatment determination unit 306. Further, when the rigidity control unit 302 determines, based on the detection result from the treatment determination unit 306, that treatment using the predetermined treatment instrument 125 is about to be performed, the rigidity control unit 302 performs control of increasing rigidity of the rigidity variable unit 112 located on a proximal end side in the “bent portion”.

In the present embodiment, each unit of the body device 30 may be an individual electronic circuit, or may be a circuit block of an integrated circuit, such as an FPGA (field programmable gate array). In the present embodiment, for example, the body device 30 may be configured to include one or more processors (CPUs or the like).

### Manner of Operation of First Embodiment

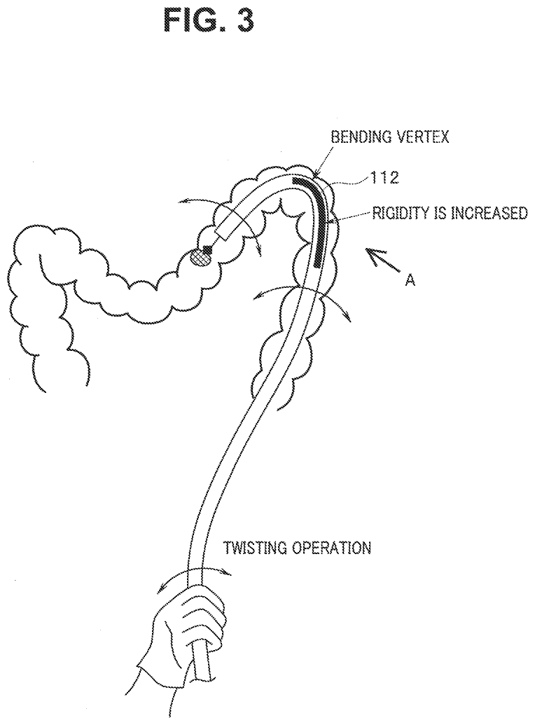

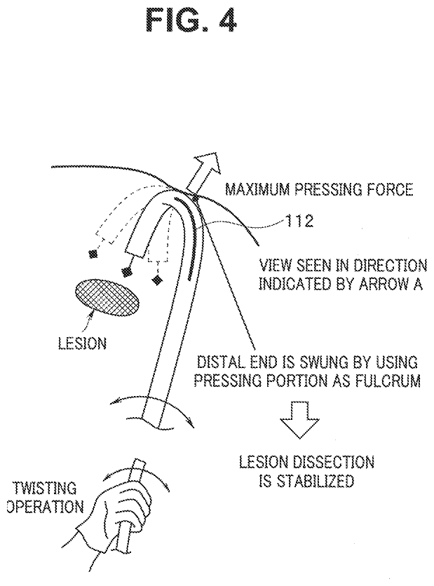

Next, in the first embodiment, the description will be made with reference to FIG. 3, FIG. 4 and FIG. 5 for a manner of operation of the rigidity control unit 302 that controls rigidity of the rigidity variable unit 112 based on signals from the shape analysis unit 304, the bent portion detection unit 305, and the treatment determination unit 306.

FIG. 3 is an explanatory view showing control of the rigidity variable unit when the bent portion occurs in the insertion portion of the endoscope of the endoscope system according to the first embodiment in a state where the insertion portion is inserted into the body cavity of the subject. FIG. 4 is an enlarged view of a main part showing a state where the bent portion of the insertion portion of the endoscope of the endoscope system according to the first embodiment is pressed against an inner wall of a body. FIG. 5 is a graph for describing a situation where a bent portion occurs in the insertion portion of the endoscope of the endoscope system according to the first embodiment.

In the present embodiment, first, the insertion shape information acquisition unit 402 of the insertion shape detection device 40 (see FIG. 2) detects magnetic fields generated from the source coil group 113 provided to the insertion portion 11. Then, the insertion shape information acquisition unit 402 calculates an insertion shape of the insertion portion 11 based on respective positions of the plurality of source coils included in the source coil group 113, the respective positions of the plurality of source coils being acquired based on magnitudes of the detected magnetic fields.

The insertion shape information acquisition unit 402 generates insertion shape information indicating the calculated insertion shape, and then outputs the insertion shape information to the body device 30. Under control from the control unit 301, the shape analysis unit 304 of the body device 30 acquires information from the insertion shape information acquisition unit 402 of the insertion shape detection device 40 (insertion shape information obtained by calculating the insertion shape of the insertion portion 11 and indicating the insertion shape), and then analyzes a shape of the insertion portion 11 (for example, a portion from the bending portion 11B to the flexible tube portion 11C).

Specifically, the shape analysis unit 304 calculates a radius of curvature of the bending insertion portion 11, and then sends the calculation result to the bent portion detection unit 305. Under control from the control unit 301, the bent portion detection unit 305 detects, based on the value of the radius of curvature of the insertion portion 11 calculated by the shape analysis unit 304, whether the bent portion occurs in the insertion portion 11.

Assume that, for example, as shown in FIG. 3, the insertion portion 11 is inserted into the large intestine of the subject within a range from the descending colon to the transverse colon, and a lesion that requires treatment performed using a treatment instrument is present in the transverse colon. In such a case, a portion of the insertion portion 11 ranging from the bending portion 11B to the flexible tube portion 11C significantly bends in the vicinity of the splenic flexure between the descending colon and the transverse colon.

In a state where the portion of the insertion portion 11 in the vicinity of the bending portion significantly bends in the body cavity of the subject as described above, the shape analysis unit 304 calculates a radius of curvature of the bending insertion portion 11 based on the shape information acquired from the insertion shape information acquisition unit 402. The shape analysis unit 304 sends the calculation result to the bent portion detection unit 305.

Under control from the control unit 301, the bent portion detection unit 305 detects, based on the value of the radius of curvature of the insertion portion 11 calculated by the shape analysis unit 304, whether the bent portion occurs in the insertion portion 11.

When the value of the radius of curvature of the insertion portion 11 calculated by the shape analysis unit 304 is equal to or less than a predetermined value, for example, as shown in FIG. 5, when a value of a radius of curvature of the bending portion 11B is small, that is, the insertion portion 11 is significantly bent, the bent portion detection unit 305 determines that the “bent portion” is formed at a portion of the bending portion of the insertion portion 11.

Specifically, as shown in FIG. 3, when a region of the insertion portion 11 ranging from the bending portion 11B to the flexible tube portion 11C significantly bends to have a bending vertex in the vicinity of the splenic flexure of the subject, the bent portion detection unit 305 determines that the “bent portion” is formed at the portion of the bending portion of the insertion portion 11, and then outputs the determination result to the rigidity control unit 302 provided downstream of the bent portion detection unit 305.

When the bent portion detection unit 305 determines that the “bent portion” is formed in the insertion portion 11, the bent portion detection unit 305 calculates a length along which rigidity variable control of the rigidity variable unit 112 is performed (see FIG. 5). Specifically, according to a bent state of the “bent portion”, that is, according to a size of the radius of curvature of the bent portion and a position where the bent portion occurs, for example, it is necessary to control a corresponding length along which rigidity variable control of the rigidity variable unit 112 is performed (a length from the proximal end portion of the bending portion). Accordingly, in the present embodiment, the bent portion detection unit 305 determines that the “bent portion” is formed in the insertion portion 11, and the bent portion detection unit 305 calculates the length along which rigidity variable control is performed.

In contrast, when the operator first inserts the insertion portion 11 into the body cavity of the subject and then inserts the predetermined treatment instrument 125 from the channel opening portion 18 toward the treatment instrument insertion channel, under control from the control unit 301, the treatment determination unit 306 determines whether treatment using the treatment instrument is about to be performed in the body cavity of the subject.

Specifically, assume that after the insertion portion 11 is inserted, the operator turns on the scope switch 122 of the operation portion 12 to perform treatment using the treatment instrument in a state where a distal end surface of the insertion portion faces a lesion present in the transverse colon, and the portion of the insertion portion 11 ranging from the bending portion 11B to the flexible tube portion 11C significantly bends in the vicinity of the splenic flexure as shown in FIG. 3.

When the treatment determination unit 306 receives an ON signal from the scope switch 122, the treatment determination unit 306 determines that the operator is about to perform treatment using the treatment instrument by inserting the treatment instrument through the treatment instrument insertion channel of the endoscope 10. Then, the treatment determination unit 306 outputs the determination result to the rigidity control unit 302 provided downstream of the treatment determination unit 306.

Next, under control from the control unit 301, the rigidity control unit 302 usually performs an action for controlling rigidity of the rigidity variable unit 112 such that flexural rigidity in the rigidity variable range of the insertion portion 11 sequentially increases in the direction from the center portion toward both end portions in the rigidity variable range, for example.

In contrast, in the first embodiment, under control from the control unit 301, the rigidity control unit 302 controls rigidity of the rigidity variable unit 112 based on signals from the bent portion detection unit 305 and the treatment determination unit 306.

That is to say, in a case where the rigidity control unit 302 receives, from the bent portion detection unit 305, a determination result that a predetermined “bent portion” is formed at a portion of the insertion portion 11, and the rigidity control unit 302 receives information relating to the above-mentioned length along which rigidity variable control is performed, when the rigidity control unit 302 receives, from the treatment determination unit 306, a determination result that treatment using the predetermined treatment instrument 125 is about to be performed, the rigidity control unit 302 performs control of increasing rigidity of the rigidity variable unit 112 located on the proximal end side in the “bent portion”.

At this point of operation, as shown in FIG. 3, control is performed such that rigidity of the rigidity variable unit 112 is increased in the region of the insertion portion 11 ranging from the bending portion 11B to the flexible tube portion 11C. Therefore, rigidity is increased on the proximal end side in the “bent portion”.

Assume that the operator performed an operation of twisting the flexible tube portion 11C of the insertion portion 11. At this point of operation, as described above, the insertion portion 11 is brought into a state where rigidity on the proximal end side in the “bent portion” is high. Accordingly, as shown in FIG. 4, a portion of the “bent portion” in the vicinity of the bending vertex is pressed against a contact point on the inner wall of the body with the twisting operation of the flexible tube portion 11C.

Thereafter, when the twisting operation is further performed on the flexible tube portion 11C of the insertion portion 11 in a state where the portion of the “bent portion” in the vicinity of the bending vertex is pressed against the contact point on the inner wall of the body, a pressing force of the “bent portion” against the inner wall of the body reaches a maximum. Further, a distal end portion of the insertion portion is swung by using the contact point on the inner wall of the body, that is, a pressing portion of the “bent portion”, as a fulcrum. Accordingly, it is possible to stably perform lesion dissection using a treatment instrument, such as an IT knife.

### Advantageous Effect of Endoscope System of First Embodiment

As described above, with the endoscope system of the first embodiment, when treatment is performed by inserting a treatment instrument through the treatment instrument insertion channel of the insertion portion, it is possible to stably operate the distal end portion of the insertion portion as intended without being affected by mucus or the like.

In the present embodiment, a magnetic sensor system is adopted for the insertion shape detection device 40. However, the insertion shape detection device 40 is not limited to such a system. A shape sensor and an insertion amount sensor may be adopted for the insertion shape detection device. Further, an ultrasonic system, an optical system, or a system that uses an acceleration sensor may be adopted for the insertion shape detection device. That is to say, it is sufficient to adopt a system that can detect a position or a relative position of the insertion portion 11 with respect to the subject or with respect to a location, such as a room where the subject is located.

Specifically, for example, the above-mentioned insertion amount sensor may include a rotation amount (torsion amount) sensor when necessary. By detecting an amount of rotation (amount of torsion) of the insertion portion inserted into the subject (patient), it is possible to acquire a relative position with respect to the subject (patient) more accurately.

### Second Embodiment

Next, a second embodiment of the present invention will be described.

An endoscope system according to the second embodiment is characterized by detecting whether the “bent portion” formed in the insertion portion is pressed against the inner wall surface of the body cavity portion of the subject, and by performing control of increasing rigidity of the rigidity variable unit located on the proximal end side in the bent portion when it is determined that the bent portion is pressed against the inner wall surface of the body cavity portion of the subject.

Other components are substantially equivalent to the corresponding components of the first embodiment and hence, in the second embodiment, only points that make the second embodiment different from the first embodiment will be described, and the description of the same components will be omitted.

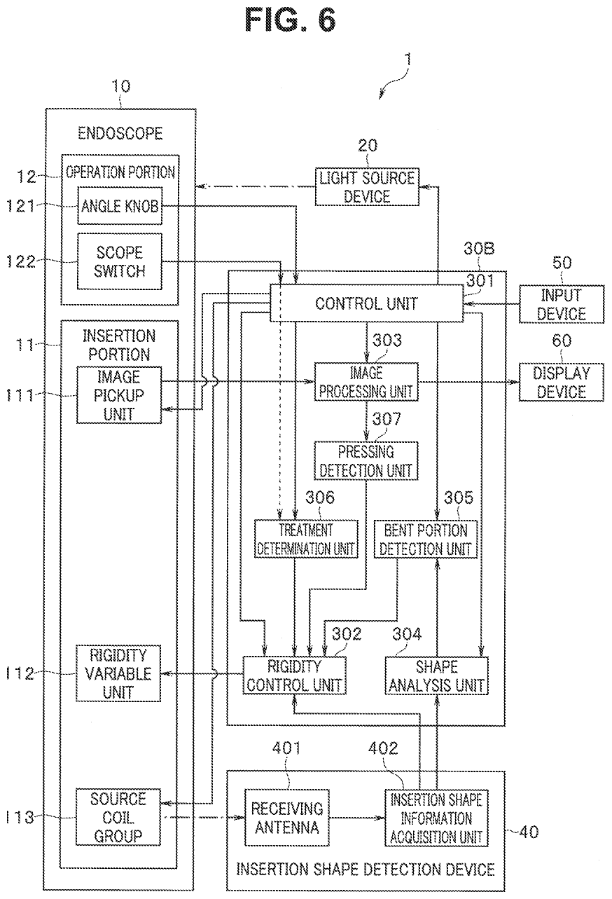

FIG. 6 is a block diagram showing an electrical configuration of the endoscope system according to the second embodiment of the present invention.

In the second embodiment, a body device 30B includes a pressing detection unit 307. In a state where the “bent portion” is formed in the insertion portion 11 as described above, the pressing detection unit 307 detects whether the bent portion is pressed against the inner wall surface of the body cavity portion of the subject.

Based on an endoscope image on which image processing is performed by the image processing unit 303, the pressing detection unit 307 detects whether the “bent portion”, which is formed in the insertion portion 11, is pressed against the inner wall surface of the body cavity portion of the subject. For example, the pressing detection unit 307 calculates a ratio in a screen between a luminal portion and the inner wall surface portion in the endoscope image by performing image processing. When a ratio of the inner wall surface is large, the pressing detection unit 307 determines that the “bent portion” is pressed against the inner wall surface. The pressing detection unit 307 sends the detection result (pressing information) to the rigidity control unit 302 provided downstream of the pressing detection unit 307.

In the second embodiment, under control from the control unit 301, the rigidity control unit 302 controls rigidity of the rigidity variable unit 112 based on signals from the bent portion detection unit 305 and the treatment determination unit 306. In addition to the above, the rigidity control unit 302 controls rigidity of the rigidity variable unit 112 based on the detection result (pressing information), which is obtained from the pressing detection unit 307.

That is to say, in the second embodiment, the rigidity control unit 302 acquires the pressing information from the pressing detection unit 307, thus being able to more accurately determine whether the “bent portion” of the insertion portion 11 is pressed against the inner wall surface of the body cavity portion of the subject. Therefore, it is possible to more accurately perform control of increasing rigidity of the rigidity variable unit 112 located on the proximal end side in the bent portion.

### Advantageous Effect of Endoscope System of Second Embodiment

In the endoscope system according to the second embodiment, the rigidity control unit 302 acquires pressing information from the pressing detection unit 307 and hence, the rigidity control unit 302 can more accurately control rigidity of the rigidity variable unit 112 located on the proximal end side in the bent portion.

### Third Embodiment

Next, a third embodiment of the present invention will be described.

An endoscope system according to the third embodiment is characterized by calculating an amount of bending of the bending portion of the insertion portion 11 based on an amount of rotation of the angle knob 121 of the operation portion 12, and by controlling rigidity of the rigidity variable unit 112 according to the amount of bending.

Other components are substantially equivalent to the corresponding components of the first embodiment and hence, in the third embodiment, only points that make the third embodiment different from the first embodiment will be described, and the description of the same components will be omitted.

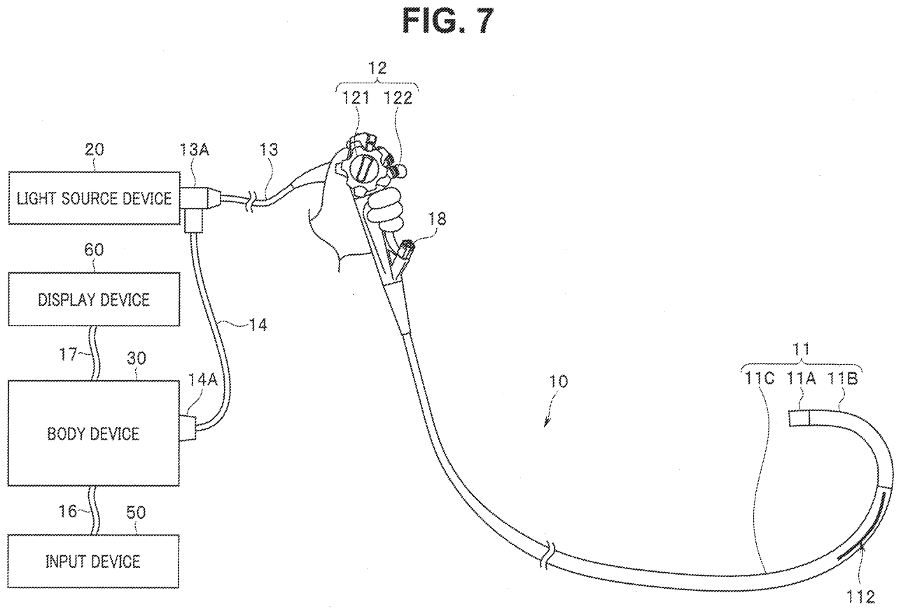

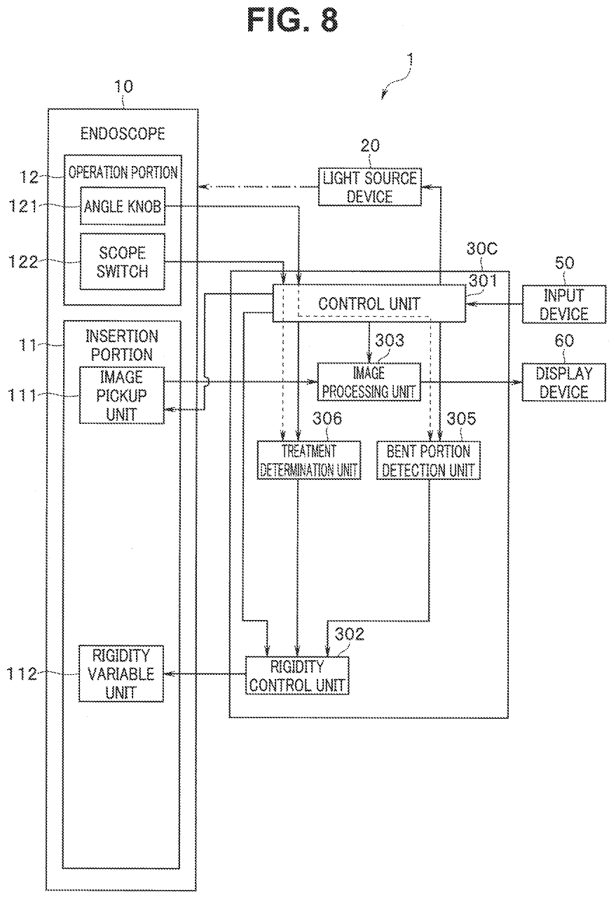

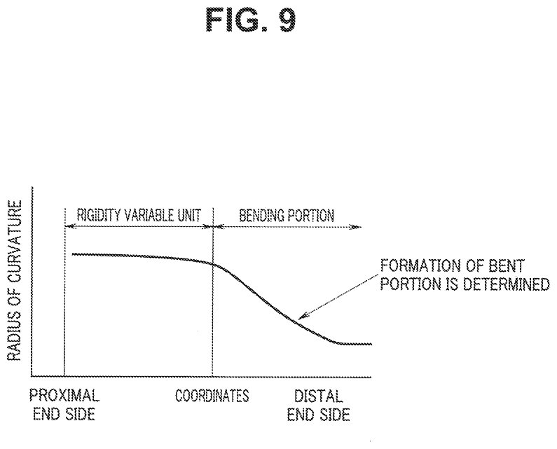

FIG. 7 is a block diagram showing a configuration of the endoscope system according to the third embodiment of the present invention. FIG. 8 is a block diagram showing an electrical configuration of the endoscope system according to the third embodiment. FIG. 9 is a graph for describing a situation where a bent portion occurs in the insertion portion of the endoscope of the endoscope system according to the third embodiment.

Unlike the first embodiment, the endoscope system according to the third embodiment has no function of a so-called endoscope insertion shape detection device (UPD). Accordingly, as shown in FIG. 7 and FIG. 8, unlike the first embodiment, the insertion shape detection device 40 and the source coil group 113 disposed in the insertion portion 11 are omitted. In addition, in the third embodiment, the shape analysis unit 304, which acquires shape information from the insertion shape information acquisition unit 402, is also omitted from a body device 30C.

In contrast, in the third embodiment, the bent portion detection unit 305 acquires information relating to an amount of rotation of the angle knob 121 of the operation portion 12. In the present embodiment, the bending portion 11B of the insertion portion 11 is configured to be able to bend according to an operation of the angle knob 121 provided to the operation portion 12.

In the present embodiment, the bent portion detection unit 305 calculates an amount of bending of the bending portion of the insertion portion 11 based on the acquired information relating to the amount of rotation of the angle knob 121 and, based on the calculation result, detects whether the bent portion occurs in the insertion portion 11.

Specifically, in a case where a value of a radius of curvature corresponding to the amount of bending of the bending portion of the insertion portion 11, which is calculated from the amount of rotation of the angle knob 121, is equal to or less than a predetermined value, for example, in a case where, as shown in FIG. 9, the bending portion significantly bends, the bent portion detection unit 305 determines that the “bent portion” is formed at a portion of the bending portion of the insertion portion 11, and then outputs the determination result to the rigidity control unit 302 provided downstream of the bent portion detection unit 305.

Other manners of operation and advantageous effects, that is, control of rigidity of the rigidity variable unit 112 performed by the rigidity control unit 302, are substantially equivalent to the corresponding manner of operation and advantageous effects of the first embodiment and hence, the description will be omitted in the third embodiment.

### Advantageous Effect of Endoscope System of Third Embodiment

As described above, with the endoscope system according to the third embodiment, even without a function of the so-called endoscope insertion shape detection device (UPD), it is possible to determine formation of the “bent portion” of the bending portion of the insertion portion 11 based on information on the amount of rotation of the angle knob 121, thus allowing the rigidity control unit 302 to control rigidity of the rigidity variable unit 112 based on this determination.

### Fourth Embodiment

Next, a fourth embodiment of the present invention will be described.

Unlike the first embodiment, an endoscope system according to the fourth embodiment is characterized in that a rigidity variable unit is provided, the rigidity variable unit being embedded in the insertion portion in a state of having a plurality of segments arranged in the longitudinal direction of the insertion portion and, at a desired position of the flexible tube portion of the insertion portion 11, rigidity of a portion on a proximal end side of the bent portion is controlled according to a bent state of the rigidity variable unit.

Other components are substantially equivalent to the corresponding components of the first embodiment and hence, in the fourth embodiment, only points that make the fourth embodiment different from the first embodiment will be described, and the description of the same components will be omitted. It is assumed that the endoscope system 1 of the fourth embodiment is an endoscope system including a so-called upper digestive tract endoscope that is to be orally inserted into the upper digestive tract of the subject, such as the esophagus, the stomach, and the duodenum.

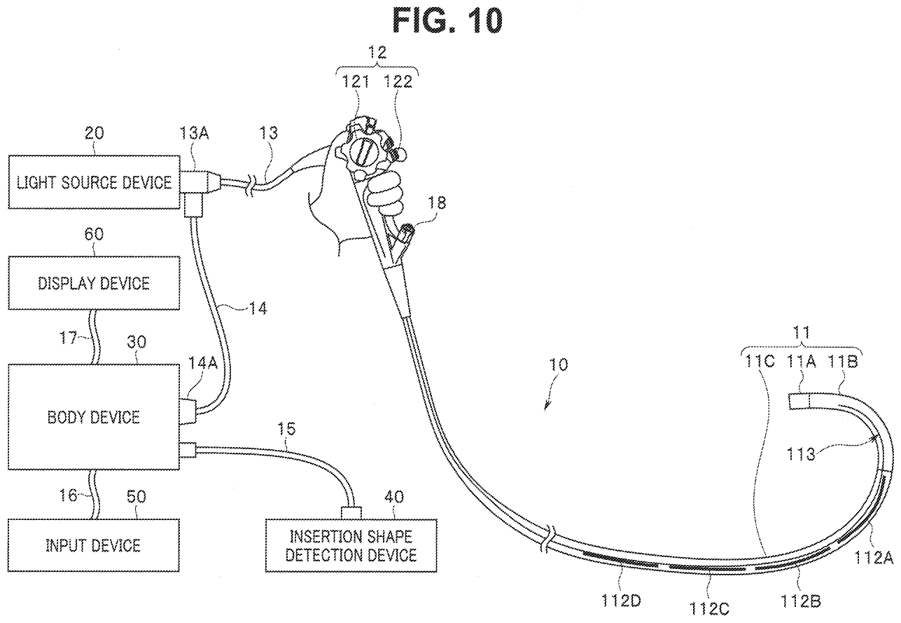

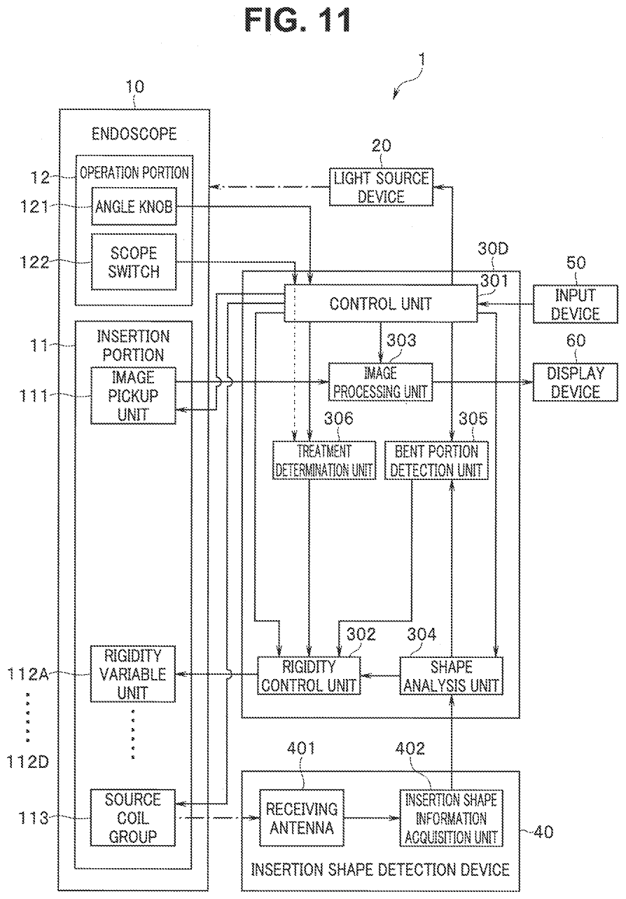

FIG. 10 is a block diagram showing a configuration of the endoscope system according to the fourth embodiment of the present invention. FIG. 11 is a block diagram showing an electrical configuration of the endoscope system according to the fourth embodiment.

As shown in FIG. 10, in the fourth embodiment, a plurality of rigidity variable units 112A, 112B, 112C, and 112D are embedded in the insertion portion 11 in the longitudinal direction of the insertion portion 11. In the same manner as the rigidity variable unit 112 in the first embodiment, each of the rigidity variable units 112A, 112B, 112C, and 112D in the fourth embodiment is formed as an actuator including a coil heater and a shape memory member not shown in the drawing. The rigidity variable units 112A, 112B, 112C, and 112D are provided in the longitudinal direction of the insertion portion 11 in a predetermined range from the proximal end portion of the bending portion 11B to the distal end portion of the flexible tube portion 11C.

In the present embodiment, the rigidity variable units 112A, 112B, 112C, and 112D are provided as four segments. However, the configuration is not limited to such a configuration. The rigidity variable units may be provided as a large number of segments. Further, the rigidity variable unit per se may have any one of various configurations which will be described later (see FIG. 23 to FIG. 28).

As shown in FIG. 11, a body device 30D in the fourth embodiment has a configuration substantially equivalent to the configuration of the body device 30 in the first embodiment. In the same manner as in the first embodiment, under control from the control unit 301, rigidity control of the above-mentioned rigidity variable units 112A, 112B, 112C, and 112D of the endoscope 10 is performed by the rigidity control unit 302 based on signals from the shape analysis unit 304, the bent portion detection unit 305, and the treatment determination unit 306.

### Manner of Operation of Fourth Embodiment

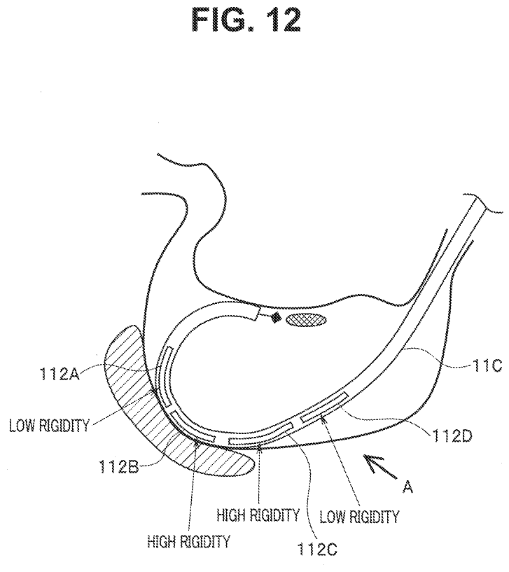

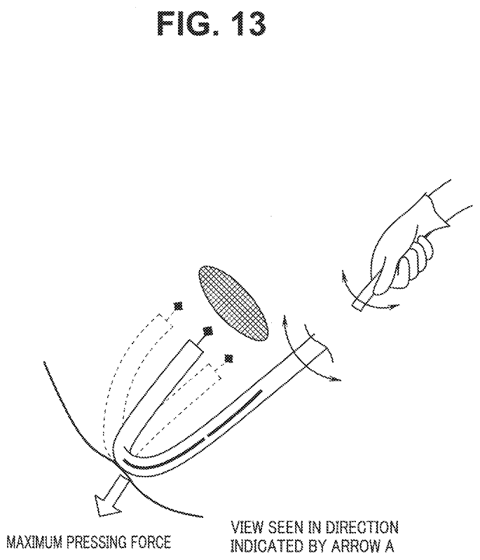

Next, in the fourth embodiment, the description will be made with reference to FIG. 12 and FIG. 13 for a manner of operation of the rigidity control unit 302 that controls rigidities of the rigidity variable units 112A, 112B, 112C, and 112D based on signals from the shape analysis unit 304, the bent portion detection unit 305, and the treatment determination unit 306.

FIG. 12 is an explanatory view showing control of the rigidity variable units when the bent portion occurs in the insertion portion of the endoscope of the endoscope system according to the fourth embodiment in a state where the insertion portion is inserted into the body cavity of the subject. FIG. 13 is an enlarged view of a main part showing a state where the bent portion of the insertion portion of the endoscope of the endoscope system according to the fourth embodiment is pressed against the inner wall of the body.

Also in the fourth embodiment, in the same manner as in the first embodiment, based on respective positions of the plurality of source coils included in the source coil group 113, the insertion shape information acquisition unit 402 of the insertion shape detection device 40 (see FIG. 11) first calculates an insertion shape of the insertion portion 11, generates insertion shape information indicating the calculated insertion shape, and then outputs the insertion shape information to the body device 30D.

Under control from the control unit 301, the shape analysis unit 304 of the body device 30D acquires information from the insertion shape information acquisition unit 402 of the insertion shape detection device 40, and then analyzes a shape of the insertion portion 11 (for example, a portion from the bending portion 11B to the flexible tube portion 11C).

Manners of operation of the shape analysis unit 304, the bent portion detection unit 305, and the treatment determination unit 306 are substantially equivalent to the corresponding manners of operation in the first embodiment. Under control from the control unit 301, the bent portion detection unit 305 detects, based on a value of a radius of curvature of the insertion portion 11 calculated by the shape analysis unit 304, whether the bent portion occurs in the insertion portion 11.

Assume that, for example, as shown in FIG. 12, the insertion portion 11 is inserted into the stomach from the cardiac region of the subject, and a lesion that requires treatment performed using a treatment instrument is present on a lesser curvature side of the gastric body. In such a case, a region of the insertion portion 11 ranging from the bending portion 11B to the flexible tube portion 11C significantly bends at parts in a range from the greater curvature side of the gastric body to the vicinity of the prepyloric region.

In a state where the region of the insertion portion 11 ranging from the bending portion 11B to the flexible tube portion 11C significantly bends in the body cavity of the subject as described above, the shape analysis unit 304 calculates radius of a curvature of the bending insertion portion 11 based on shape information acquired from the insertion shape information acquisition unit 402. The shape analysis unit 304 sends the calculation result to the bent portion detection unit 305.

Also in the fourth embodiment, under control from the control unit 301, the bent portion detection unit 305 detects, based on the value of the radius of curvature of the insertion portion 11 calculated by the shape analysis unit 304, whether the bent portion occurs in the insertion portion 11.

When the value of the radius of curvature of the insertion portion 11 calculated by the shape analysis unit 304 is equal to or less than a predetermined value, for example, when a value of a radius of curvature of the bending portion 11B is small, that is, the insertion portion 11 is significantly bent, in the same manner as in the first embodiment, the bent portion detection unit 305 determines that the “bent portion” is formed at a portion of the bending portion of the insertion portion 11.

Specifically, as shown in FIG. 12, in a case where the region of the insertion portion 11 ranging from the bending portion 11B to the flexible tube portion 11C significantly bends to have a bending vertex at parts in a range from the greater curvature side of the gastric body to the vicinity of the prepyloric region of the subject, the bent portion detection unit 305 determines that the “bent portion” is formed at a portion of the bending portion of the insertion portion 11, and then outputs the determination result to the rigidity control unit 302 provided downstream of the bent portion detection unit 305.

In contrast, also in the fourth embodiment, when the operator first inserts the insertion portion 11 into the body cavity of the subject and then inserts the predetermined treatment instrument 125 from the channel opening portion 18 toward the treatment instrument insertion channel, under control from the control unit 301, the treatment determination unit 306 determines whether treatment using the treatment instrument is about to be performed in the body cavity of the subject.

Specifically, assume that after the insertion portion 11 is inserted, the operator turns on the scope switch 122 of the operation portion 12 to perform treatment using the treatment instrument in a state where a distal end surface of the insertion portion faces a lesion present on the lesser curvature side of the gastric body, and the region of the insertion portion 11 ranging from the bending portion 11B to the flexible tube portion 11C significantly bends at the parts in a range from the greater curvature side of the gastric body to the vicinity of the prepyloric region as shown in FIG. 12.

When the treatment determination unit 306 receives an ON signal from the scope switch 122, the treatment determination unit 306 determines that the operator is about to perform treatment using the treatment instrument by inserting the treatment instrument through the treatment instrument insertion channel of the endoscope 10. Then, the treatment determination unit 306 outputs the determination result to the rigidity control unit 302 provided downstream of the treatment determination unit 306.

Next, also in the fourth embodiment, the rigidity control unit 302 controls rigidities of the rigidity variable units 112A, 112B, 112C, and 112D based on signals from the bent portion detection unit 305 and the treatment determination unit 306.

That is to say, in a case where the rigidity control unit 302 receives, from the bent portion detection unit 305, a determination result that a predetermined “bent portion” is formed at a portion of the insertion portion 11, when the rigidity control unit 302 receives, from the treatment determination unit 306, a determination result that treatment using the predetermined treatment instrument 125 is about to be performed, the rigidity control unit 302 performs rigidity control of increasing or decreasing rigidity of each of the rigidity variable units 112A, 112B, 112C, and 112D located on the proximal end side in the “bent portion”.

Specifically, the rigidity control unit 302 independently controls rigidities of the respective rigidity variable units 112A, 112B, 112C, and 112D, which are arranged in this order from the distal end side in the region of the insertion portion 11 ranging from the bending portion 11B to the flexible tube portion 11C as shown in FIG. 12.

That is to say, of the rigidity variable units 112A, 112B, 112C, and 112D, rigidities of the rigidity variable unit 112B and the rigidity variable unit 112C are increased, the rigidity variable unit 112B and the rigidity variable unit 112C corresponding to a region where the “bent portion” is formed in the region of the insertion portion 11 ranging from the bending portion 11B to the flexible tube portion 11C. In contrast, rigidities of the rigidity variable unit 112A and the rigidity variable unit 112D are decreased, the rigidity variable unit 112A and the rigidity variable unit 112D corresponding to a region outside the “bent portion”. With such operations, it is possible to relatively increase the rigidities of the rigidity variable unit 112B and the rigidity variable unit 112C and hence, it is possible to press the “bent portion” against the inner wall of the body more effectively (see FIG. 13).

Also in the fourth embodiment, when the operator performs an operation of twisting the flexible tube portion 11C of the insertion portion 11, a pressing force of the “bent portion” against the inner wall of the body reaches a maximum. Further, the distal end portion of the insertion portion is swung by using the contact point on the inner wall of the body, that is, a pressing portion of the “bent portion”, as a fulcrum. Accordingly it is possible to stably perform lesion dissection using a treatment instrument, such as an IT knife.

### Advantageous Effect of Endoscope System of Fourth Embodiment

As described above, with the endoscope system of the fourth embodiment, in the same manner as in the first embodiment, when treatment is performed by inserting a treatment instrument through the treatment instrument insertion channel of the insertion portion, it is possible to stably operate the distal end portion of the insertion portion as intended without being affected by mucus or the like, and it is also possible to accurately perform rigidity control at any position in the insertion portion with a greater range.

### Fifth Embodiment

Next, a fifth embodiment of the present invention will be described.

An endoscope system according to the fifth embodiment is characterized by detecting, without depending on a switching operation (trigger) performed by the operator, that treatment using a treatment instrument is to be performed.

Other components are substantially equivalent to the corresponding components of the first embodiment and hence, in the fifth embodiment, only points that make the fifth embodiment different from the first embodiment will be described, and the description of the same components will be omitted.

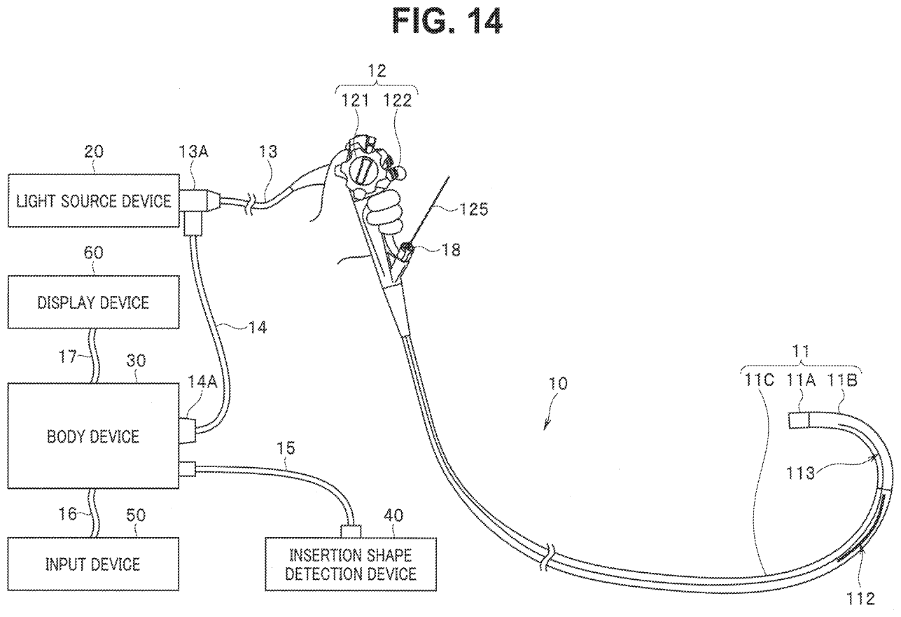

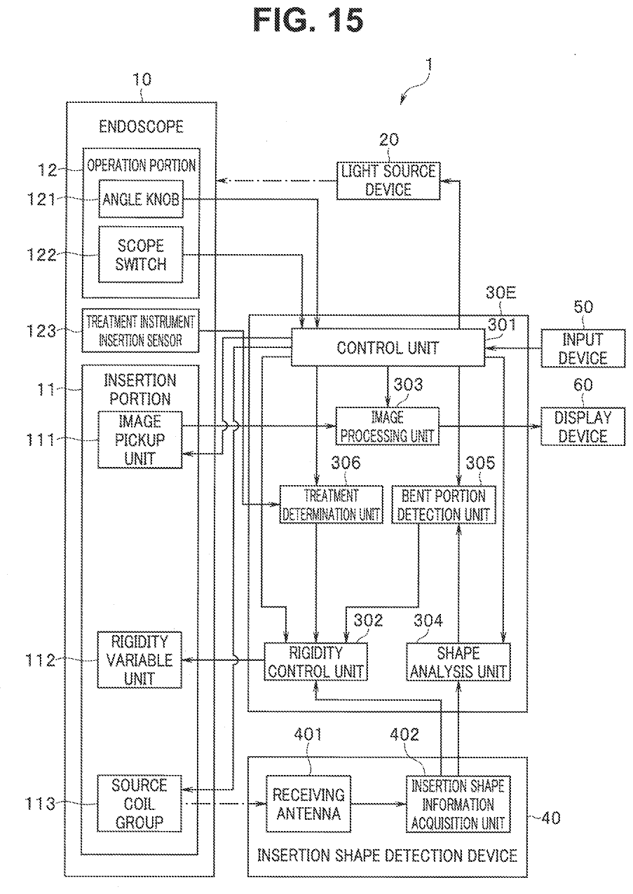

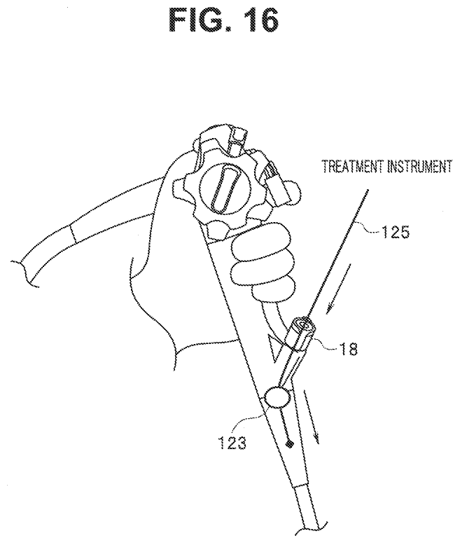

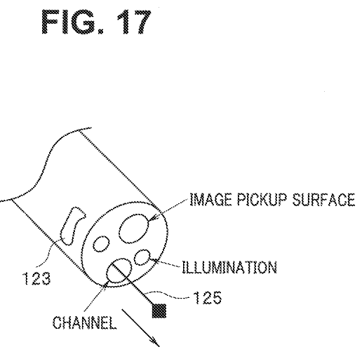

FIG. 14 is a block diagram showing a configuration of the endoscope system according to the fifth embodiment of the present invention. FIG. 15 is a block diagram showing an electrical configuration of the endoscope system according to the fifth embodiment. FIG. 16 is an enlarged view of a main part showing a treatment instrument insertion sensor of the endoscope system according to the fifth embodiment. FIG. 17 is an enlarged perspective view of a main part showing the treatment instrument insertion sensor of the endoscope system according to the fifth embodiment.

As shown in FIG. 14, FIG. 15, and FIG. 16, in the fifth embodiment, a treatment instrument insertion sensor 123 is disposed at a position near the channel opening portion 18. The treatment instrument insertion sensor 123 detects insertion of the treatment instrument 125 into the treatment instrument insertion channel.

As shown in FIG. 17, the treatment instrument insertion sensor 123 is disposed on an outer peripheral surface side of the treatment instrument insertion channel in the channel opening portion 18. The treatment instrument insertion sensor 123 is a sensor that detects passing of the treatment instrument 125 when the treatment instrument 125 is inserted from the channel opening portion 18 toward the treatment instrument insertion channel.

In the fifth embodiment, the treatment determination unit 306 provided to a body device 30E is configured to receive an output signal from the treatment instrument insertion sensor 123. Also in the fifth embodiment, under control from the control unit 301, the treatment determination unit 306 determines whether predetermined treatment is about to be performed by the operator.

That is to say, in the fifth embodiment, when the operator first inserts the insertion portion 11 into the body cavity of the subject and then inserts the predetermined treatment instrument 125 from the channel opening portion 18 toward the treatment instrument insertion channel, based on an output signal from the treatment instrument insertion sensor 123, the treatment determination unit 306 determines whether treatment using the treatment instrument is about to be performed in the body cavity of the subject.

Specifically, in the fifth embodiment, when the treatment determination unit 306 receives an ON signal from the treatment instrument insertion sensor 123, that is, when the treatment determination unit 306 receives information about insertion of the treatment instrument 125 from the channel opening portion 18 toward the treatment instrument insertion channel, the treatment determination unit 306 determines that treatment using the treatment instrument is about to be performed.

Other manners of operation and advantageous effects, that is, control of rigidity of the rigidity variable unit 112 performed by the rigidity control unit 302, are substantially equivalent to the corresponding manners of operation and advantageous effects of the first embodiment and hence, the description will be omitted in the fifth embodiment.

### Advantageous Effect of Endoscope System of Fifth Embodiment

As described above, with the endoscope system according to the fifth embodiment, it is possible to surely detect, without depending on a switching operation (trigger) performed by the operator, that treatment using the treatment instrument is to be performed.

### Sixth Embodiment

Next, a sixth embodiment of the present invention will be described.

In the same manner as the fifth embodiment, an endoscope system according to the sixth embodiment is also characterized by detecting, without depending on a switching operation (trigger) performed by the operator, that treatment using a treatment instrument is to be performed.

Other components are substantially equivalent to the corresponding components of the first embodiment and hence, in the sixth embodiment, only points that make the sixth embodiment different from the first embodiment will be described, and the description of the same components will be omitted.

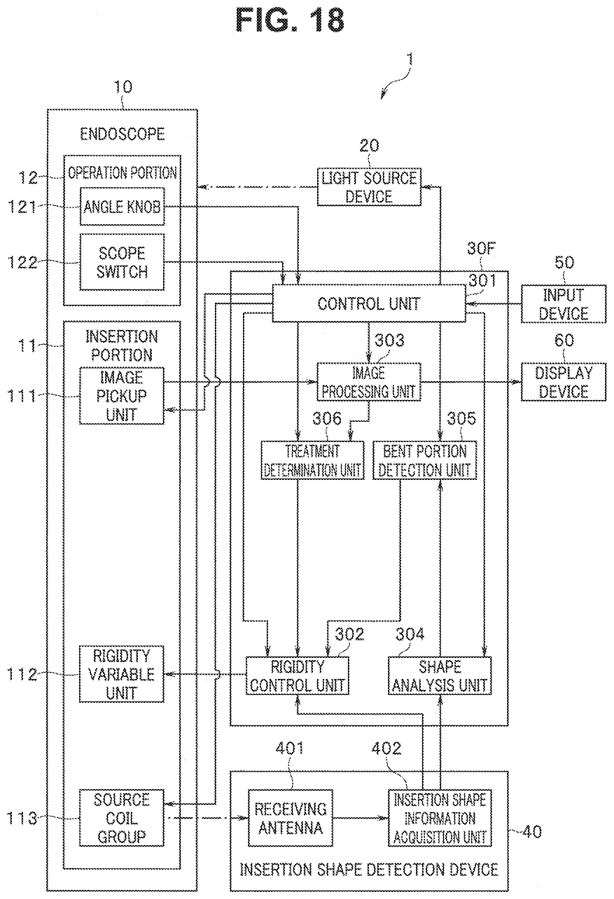

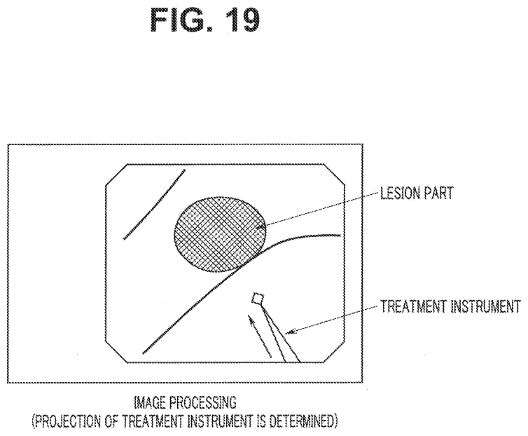

FIG. 18 is a block diagram showing an electrical configuration of the endoscope system according to the sixth embodiment of the present invention. FIG. 19 is a view for describing determination made by the treatment determination unit of the endoscope system according to the sixth embodiment after the treatment determination unit receives image processing information.

As shown in FIG. 18, in the sixth embodiment, the treatment determination unit 306 provided to a body device 30F acquires a signal relating to an endoscope image from the image processing unit 303. In the sixth embodiment, by determining, based on the acquired endoscope image (see FIG. 19), whether the treatment instrument 125 reaches a position near a target lesion, the treatment determination unit 306 determines whether predetermined treatment is about to be performed by the operator.

That is to say, in the sixth embodiment, after the operator first inserts the insertion portion 11 into the body cavity of the subject and then inserts the predetermined treatment instrument 125 from the channel opening portion 18 toward the treatment instrument insertion channel, appearance of a distal end portion of the treatment instrument 125 on a screen showing a lesion part is detected by a predetermined image processing technique (see FIG. 19). Based on a result of the image recognition, the treatment determination unit 306 determines whether treatment using the treatment instrument is about to be performed in the body cavity of the subject.

Other manners of operation and advantageous effects, that is, control of rigidity of the rigidity variable unit 112 performed by the rigidity control unit 302, are substantially equivalent to the corresponding manners of operation and advantageous effects of the first embodiment and hence, the description will be omitted in the sixth embodiment.

### Advantageous Effect of Endoscope System of Sixth Embodiment

As described above, with the endoscope system according to the sixth embodiment, it is possible to surely detect, without depending on a switching operation (trigger) performed by the operator, that treatment using the treatment instrument is to be performed.

### Seventh Embodiment

Next, a seventh embodiment of the present invention will be described.

An endoscope system according to the seventh embodiment is characterized by controlling rigidity of the rigidity variable unit such that there is small displacement in a positional relationship of an image of a distal end of a treatment instrument inserted through the treatment instrument insertion channel with respect to a target image (lesion) in an endoscope image.

Other components are substantially equivalent to the corresponding components of the first embodiment and hence, in the seventh embodiment, only points that make the seventh embodiment different from the first embodiment will be described, and the description of the same components will be omitted.

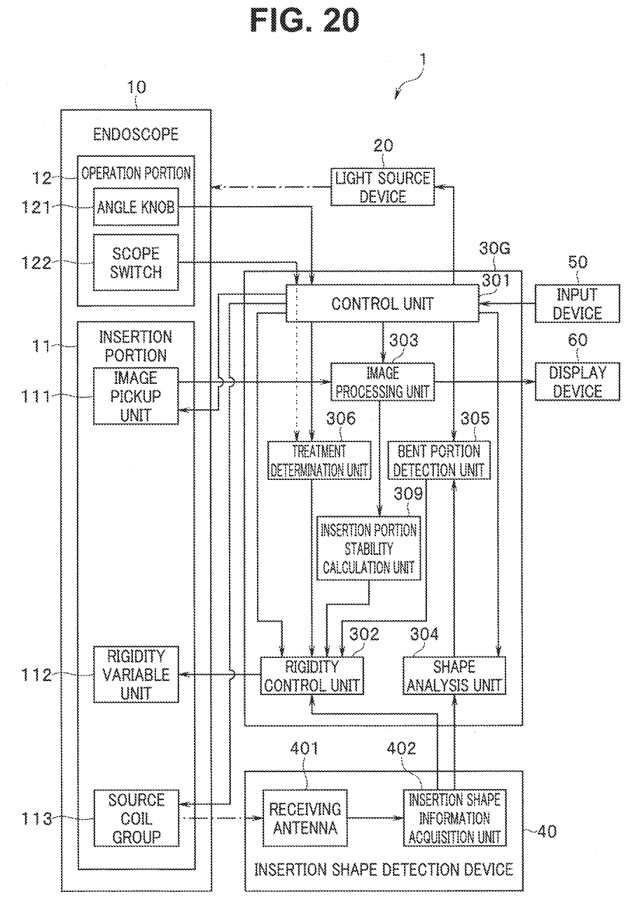

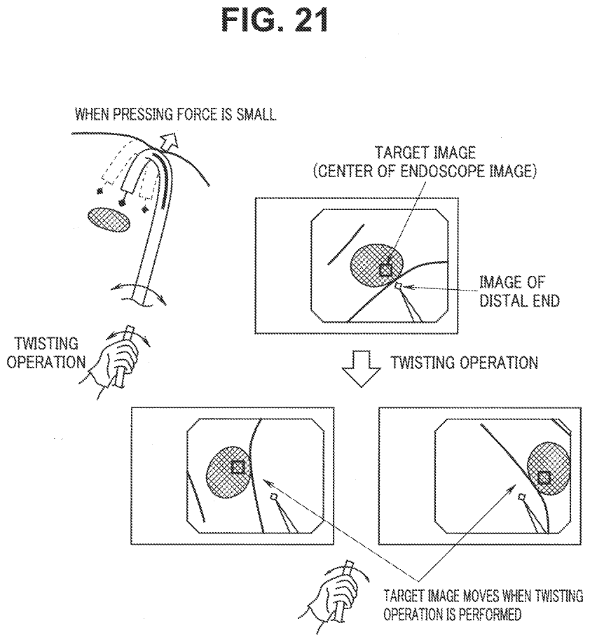

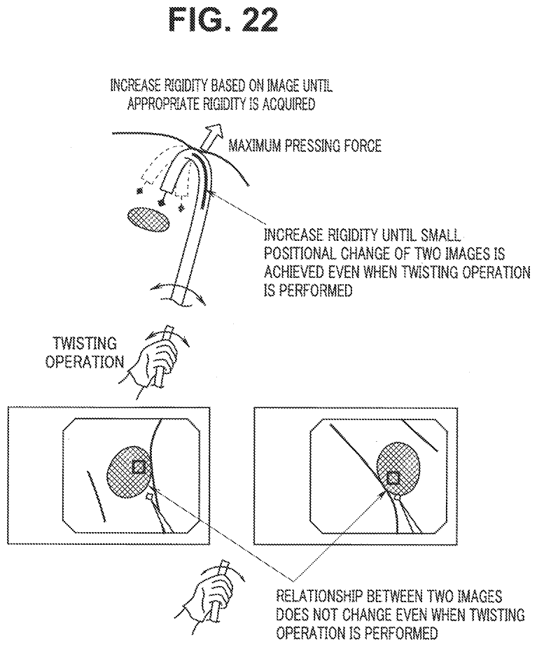

FIG. 20 is a view for describing a manner of operation of an insertion portion stability calculation unit of the endoscope system according to the seventh embodiment of the present invention. FIG. 21 is a view for describing the manner of operation of the insertion portion stability calculation unit of the endoscope system according to the seventh embodiment. FIG. 22 is a view for describing the manner of operation of the insertion portion stability calculation unit of the endoscope system according to the seventh embodiment.

As shown in FIG. 20, a body device 30G of the endoscope system of the seventh embodiment includes an insertion portion stability calculation unit 309 that calculates stability of the insertion portion 11 at the time of the insertion portion 11 being inserted into the body cavity of the subject.

In a state where the insertion portion 11 is inserted into the body cavity of the subject, the insertion portion stability calculation unit 309, first, determines whether a twisting action is performed while the insertion portion 11 is pressed against the inner wall of the body cavity portion of the subject. For example, the insertion portion stability calculation unit 309 acquires an endoscope image from the image processing unit 303, and then determines the presence or absence of the twisting action based on the acquired endoscope image, that is, based on information on movement of feature points in the endoscope image showing a target image (lesion) and an image of the distal end of the treatment instrument inserted through the treatment instrument insertion channel. Then, when the insertion portion stability calculation unit 309 determines that the twisting operation is performed on the insertion portion 11 by the operator, the insertion portion stability calculation unit 309 calculates a degree of displacement in a relative positional relationship of the image of the distal end of the treatment instrument with respect to the target image (lesion).

The above-mentioned determination of the presence or absence of the twisting action is not limited to determination based on an analysis of an endoscope image. For example, the presence or absence of the twisting action may be determined based on information from the shape sensor provided to the insertion portion 11. In this case, when a shape rapidly changes, it is possible to determine that the twisting action is performed while the insertion portion 11 is pressed against the inner wall.

In the seventh embodiment, a degree of displacement in the relative positional relationship is taken as “stability” of the insertion portion 11. Specifically, in a case where displacement in the relative positional relationship is large, that is, in a case where there is large displacement in the relative positional relationship of the image of the distal end of the treatment instrument with respect to the target image (lesion) when the operator performs the twisting operation on the insertion portion 11, it is possible to say that stability of the insertion portion 11 is low. However, in the present embodiment, an inverse of the displacement is taken as a value of stability.

In contrast, in the seventh embodiment, the rigidity control unit 302 obtains a calculation result of the value of the above-mentioned “stability” calculated by the insertion portion stability calculation unit 309. Based on the calculation result, in a case where the value of “stability” of the insertion portion 11 is less than a predetermined value (that is, in a case where there is large displacement in the relative positional relationship of the image of the distal end of the treatment instrument with respect to the target image (lesion) when the operator performs the twisting operation on the insertion portion 11), the rigidity control unit 302 continues control of increasing rigidity of the rigidity variable unit 112. In a case where the value of “stability” of the insertion portion 11 is equal to or greater than the predetermined value (that is, in a case where there is small displacement in the relative positional relationship of the image of the distal end of the treatment instrument with respect to the target image (lesion) when the operator performs the twisting operation on the insertion portion 11), the rigidity control unit 302 stops control of increasing rigidity of the rigidity variable unit 112.

Specifically, as shown in FIG. 21, in a case where the twisting operation of the insertion portion 11 is performed when a pressing force of the “bent portion”, which is formed in the insertion portion 11, against the inner wall of the body cavity portion is small, in the endoscope image from the image processing unit 303 (the endoscope image showing the target image (lesion) and the image of the distal end of the treatment instrument inserted through the treatment instrument insertion channel), the target image (lesion) significantly moves with respect to the image of the distal end of the treatment instrument. There is large displacement in the relative positional relationship of the image of the distal end of the treatment instrument with respect to the target image (lesion), that is, it is possible to say that “stability” of the insertion portion 11 is low.

When “stability” of the insertion portion 11 is low as described above, the rigidity control unit 302 in the seventh embodiment performs control of increasing rigidity of the rigidity variable unit 112.

In a case where rigidity of the rigidity variable unit 112 is increased due to control from the rigidity control unit 302, thus increasing a pressing force of the “bent portion”, which is formed in the insertion portion 11, against the inner wall of the body cavity portion, as shown in FIG. 22, even when the twisting operation of the insertion portion 11 is performed, there is small displacement in the relative positional relationship of the image of the distal end of the treatment instrument with respect to the target image (lesion), that is, “stability” of the insertion portion 11 is increased.

When “stability” of the insertion portion 11 is increased as described above, the rigidity control unit 302 stops control of increasing rigidity to prevent a further increase in rigidity of the rigidity variable unit 112.

Further, as shown in FIG. 22, in the seventh embodiment, by monitoring the endoscope image during the twisting operation of the insertion portion 11, the insertion portion stability calculation unit 309 and the rigidity control unit 302 can also control rigidity of the rigidity variable unit 112 such that the rigidity is increased until displacement in the relative positional relationship of the image of the distal end of the treatment instrument with respect to the target image (lesion) becomes small.

### Advantageous Effect of Endoscope System of Seventh Embodiment

As described above, in the endoscope system according to the seventh embodiment, rigidity of the rigidity variable unit is controlled such that there is small displacement in the positional relationship of the image of the distal end of the treatment instrument inserted through the treatment instrument insertion channel with respect to the target image (lesion) in the endoscope image. Accordingly, it is possible to surely stabilize treatment performed using the treatment instrument. Further, it is possible to avoid increasing rigidity more than necessary and hence, it is possible to alleviate burden on the subject.

Next, the description will be made for other configuration examples each of which is applicable for the above-mentioned rigidity variable unit 112 of the endoscope system of any one of the first to seventh embodiments. In the examples described below, six configuration examples are given as examples each of which is applicable for the rigidity variable unit 112. These configuration examples are respectively described as <configuration examples 1 to 6 of rigidity control system>.

### Configuration Example 1 of Rigidity Control System

FIG. 23 shows a rigidity variable device 210 and a rigidity control circuit 250 which constitute another configuration example applicable for the rigidity variable unit 112.

As shown in FIG. 23, the rigidity variable device 210 has a function of providing different rigidity to the flexible tube portion 11C by allowing the flexible tube portion 11C to take different rigidity states. The rigidity variable device 210 includes a shape memory member 220 and a plurality of inducing members 230. A phase of the shape memory member 220 may shift between a first phase and a second phase. The plurality of inducing members 230 cause phase shift in the shape memory member 220 between the first phase and the second phase.

When the shape memory member 220 is in the first phase, the shape memory member 220 takes a flexible state where the shape memory member 220 can easily deform according to an external force, that is, the shape memory member 220 has a low elastic modulus. Accordingly, the shape memory member 220 provides relatively low rigidity to the flexible tube portion 11C. When the shape memory member 220 is in the second phase, the shape memory member 220 takes a rigid state where the shape memory member 220 tends to have, against an external force, a memory shape that is memorized in advance, that is, the shape memory member 220 has a high elastic modulus. Accordingly, the shape memory member 220 provides relatively high rigidity to the flexible tube portion 11C.

Each inducing member 230 has the ability to generate heat. The shape memory member 220 has the property of the phase of the shape memory member 220 shifting from the first phase to the second phase due to heating of the inducing member 230.

The shape memory member 220 has an elongated shape. The plurality of inducing members 230 are arranged at intervals along a longitudinal axis of the shape memory member 220.

The shape memory member 220 may be made of a shape memory alloy, for example. Although not limited, the shape memory alloy may be an alloy containing NiTi, for example. Further, the shape memory member 220 is not limited to the above, and may be made of another material, such as shape memory polymer, shape memory gel, or shape memory ceramic.

The inducing member 230 may be a heater, for example. That is to say, the inducing member 230 may have the property of generating heat in response to supply of an electric current flowing through the inducing member 230. The inducing member 230 may be, for example, a heating wire, that is, a conductive member having large electric resistance. It is sufficient that the inducing member 230 has the ability to generate heat. The inducing member 230 is not limited to a heater, and may be an image pickup device, a light guide, or another element or another member, for example. Further, the inducing member 230 may be a structure that generates heat due to a chemical reaction.

The shape memory member 220 may be made of a conductive material. For example, an insulating film 242 is provided around the shape memory member 220. The insulating film 242 has a function of preventing short circuit between the shape memory member 220 and the inducing member 230.

The inducing member 230 may be made of a conductive material. For example, an insulating film 244 is provided around the inducing member 230. The insulating film 244 has a function of preventing short circuit between the shape memory member 220 and the inducing member 230 and short circuit between adjacent portions of the inducing member 230.

The rigidity control circuit 250 includes a plurality of drive circuits 252 that respectively drive the plurality of inducing members 230. Each drive circuit 252 includes a power supply 254 and a switch 256. Each drive circuit 252 is electrically connected with both ends of the corresponding inducing member 230. Each drive circuit 252 supplies an electric current to the corresponding inducing member 230 in response to the turning on of the switch 256, that is, in response to a closing action of the switch 256. Further, each drive circuit 252 stops supply of an electric current to the corresponding inducing member 230 in response to the turning off of the switch 256, that is, in response to an opening action of the switch 256. The inducing member 230 generates heat in response to supply of an electric current.

The shape memory member 220 may have a wire shape. The inducing members 230 are disposed at positions near the shape memory member 220. Each inducing member 230 may have a coil shape, and the shape memory member 220 may extend through the inducing members 230 having a coil shape.

When the switch 256 of the drive circuit 252 is in an off state, the shape memory member 220 is in the first phase, that is, in a flexible state having a low elastic modulus. When the shape memory member 220 is in the first phase, the shape memory member 220 is in a state of easily deforming according to an external force.

When the switch 256 of the drive circuit 252 is switched to an on state, an electric current flows through the inducing member 230, so that the inducing member 230 generates heat. As a result, the shape memory member 220 shifts to the second phase, that is, to the rigid state having a high elastic modulus. When the shape memory member 220 is in the second phase, the shape memory member 220 tends to take a memory shape.

When the shape memory member 220 is in the first phase, the rigidity variable device 210 provides relatively low rigidity to the flexible tube portion 11C, and easily deforms according to an external force acting on the flexible tube portion 11C, that is, according to a force that can deform the shape memory member 220.

When the shape memory member 220 is in the second phase, the rigidity variable device 210 provides relatively high rigidity to the flexible tube portion 11C, and tends to return to a memory shape against an external force acting on the flexible tube portion 11C, that is, against a force that can deform the shape memory member 220.

For example, when a phase of a portion of the shape memory member 220 located at a position near each inducing member 230 is switched between the first phase and the second phase by the rigidity control circuit 250, rigidity of the flexible tube portion 11C is switched. Supply of an electric current to each of the plurality of inducing members 230 can be independently switched by rigidity control circuit 250 and hence, phases of a plurality of portions of the shape memory member 220 can be independently switched. Accordingly, rigidities of the plurality of portions of the flexible tube portion 11C can be independently switched. With such a configuration, the rigidity variable device 210 can provide desired complicated rigidity distribution to the flexible tube portion 11C.

### Configuration Example 2 of Rigidity Control System

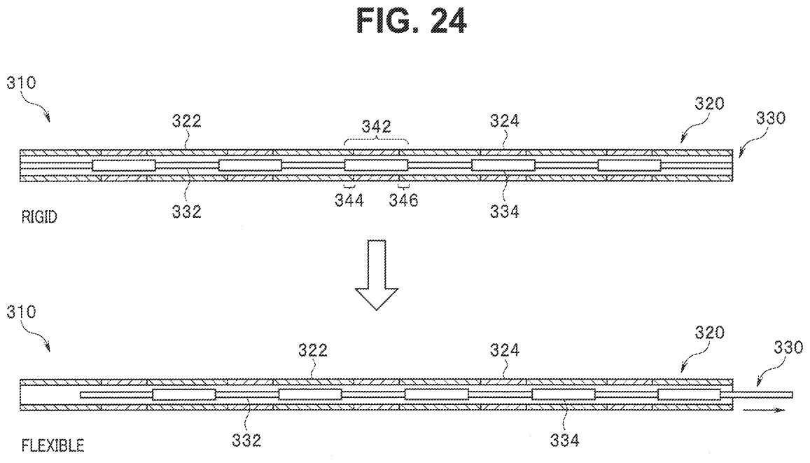

FIG. 24 shows a rigidity variable device 310 which is another configuration example applicable for the rigidity variable unit 112, and FIG. 24 shows switching of rigidity of the rigidity variable device 310 from a high rigidity state to a low rigidity state. The rigidity variable device 310 in the high rigidity state is shown at the top of FIG. 24, and the rigidity variable device 310 in the low rigidity state is shown at the bottom of FIG. 24.

The rigidity variable device 310 is a device that provides different rigidity to the flexible tube portion 11C, being an object on which the rigidity variable device 310 is mounted. The rigidity variable device 310 includes a first longitudinal member 320 and a second longitudinal member 330. The second longitudinal member 330 is disposed adjacent to and along the first longitudinal member 320. For example, the first longitudinal member 320 is an outer pipe, and the second longitudinal member 330 is a core member disposed in the outer pipe. For example, the outer pipe has an annular shape in cross section taken along a line perpendicular to an axis of the outer pipe. An outer periphery of the core member has a circular shape in cross section taken along a line perpendicular to an axis of the core member. In this case, stable flexural rigidity is provided against bending in any direction.

The first longitudinal member 320 includes a plurality of high flexural rigidity portions 322 and a plurality of low flexural rigidity portions 324. For example, the first longitudinal member 320 includes six high flexural rigidity portions 322 and five low flexural rigidity portions 324. The high flexural rigidity portions 322 and the low flexural rigidity portions 324 are arranged alternately and continuously along an axis of the first longitudinal member 320. The high flexural rigidity portion 322 has higher flexural rigidity than flexural rigidity of the low flexural rigidity portion 324. Therefore, the first longitudinal member 320 relatively easily bends at the low flexural rigidity portion 324, but does not relatively easily bend at the high flexural rigidity portion 322.

The second longitudinal member 330 includes a plurality of non-flexure-restricting portions 332 and a plurality of flexure-restricting portions 334. For example, the second longitudinal member 330 includes six non-flexure-restricting portions 332 and five flexure-restricting portions 334. The non-flexure-restricting portions 332 and the flexure-restricting portions 334 are arranged alternately and continuously along an axis of the second longitudinal member 330. The flexure-restricting portion 334 has higher flexural rigidity than flexural rigidity of the non-flexure-restricting portion 332. Therefore, the second longitudinal member 330 relatively easily bends at the non-flexure-restricting portion 332, but does not relatively easily bend at the flexure-restricting portion 334. For example, the non-flexure-restricting portion 332 is a small diameter portion having a relatively small diameter. The flexure-restricting portion 334 is a large diameter portion having a relatively large diameter. The flexure-restricting portion 334 has a constant diameter from an end portion to an end portion on an opposite side, for example.

In the rigidity variable device 310, by changing a relative position of the second longitudinal member 330 with respect to the first longitudinal member 320, it is possible to switch between a high rigidity state and a low rigidity state. In the high rigidity state, flexural rigidity of the rigidity variable device at the low flexural rigidity portions 324 is relatively high. In the low rigidity state, flexural rigidity of the rigidity variable device at the low flexural rigidity portions 324 is relatively low.

Switching from the high rigidity state to the low rigidity state is performed by moving the second longitudinal member 330 relative to the first longitudinal member 320 along the axis of the first longitudinal member 320.

In the high rigidity state, each flexure-restricting portion 334 of the second longitudinal member 330 is disposed in a range containing the low flexural rigidity portion 324 of the first longitudinal member 320. The flexure-restricting portions 334 restrict bending of the low flexural rigidity portions 324 of the first longitudinal member 320. As described above, when the second longitudinal member 330 restricts bending of the first longitudinal member 320, the rigidity variable device 310 is brought into the high rigidity state, that is, into a rigid state.

In the low rigidity state, each non-flexure-restricting portions 332 of the second longitudinal member 330 is disposed in a range containing the low flexural rigidity portion 324 of the first longitudinal member 320. The non-flexure-restricting portions 332 have a lower degree of restriction in bending of the low flexural rigidity portions 324 of the first longitudinal member 320 than the flexure-restricting portions 334. Therefore, the rigidity variable device 310 is brought into a low rigidity state where the rigidity variable device 310 easily bends at the low flexural rigidity portions 324, that is, into a flexible state.

In other words, the first longitudinal member 320 includes restricted portions 342 where bending is restricted by the flexure-restricting portions 334 in the high rigidity state. Each restricted portion 342 includes a portion 344 of a first high flexural rigidity portion 322 of the first longitudinal member 320, the low flexural rigidity portion 324, and a portion 346 of a second high flexural rigidity portion 322. The low flexural rigidity portion 324 is disposed adjacent to the first high flexural rigidity portion 322. The low flexural rigidity portion 324 is sandwiched between the first high flexural rigidity portion 322 and the portion 346 of the second high flexural rigidity portion 322. In other words, each restricted portion 342 includes the low flexural rigidity portion 324, the portion 344 of the high flexural rigidity portion 322, and the portion 346 of the high flexural rigidity portion 322. The portion 344 of the high flexural rigidity portion 322 is located on one side of the low flexural rigidity portion 324, for example, on a left side of the low flexural rigidity portion 324 in FIG. 24. The portion 346 of the high flexural rigidity portion 322 is located on the other side of the low flexural rigidity portion 324, for example, on a right side of the low flexural rigidity portion 324 in FIG. 24. A length of the restricted portion 342, that is, a dimension of the restricted portion 342 along the axis of the first longitudinal member 320 is equal to a length of the flexure-restricting portion 334, that is, a dimension of the flexure-restricting portion 334 along the axis of the second longitudinal member 330.

When the flexure-restricting portions 334 are disposed at positions corresponding to the restricted portions 342, the flexure-restricting portions 334 restrict bending of the low flexural rigidity portions 324. In contrast, when the non-flexure-restricting portions 332 are disposed at positions corresponding to the restricted portions 342, the non-flexure-restricting portions 332 provide a lower restriction on bending of the low flexural rigidity portions 324 than when the flexure-restricting portions 334 are disposed at the positions corresponding to the restricted portions 342. Accordingly, when the flexure-restricting portions 334 are disposed at the positions corresponding to the restricted portions 342, flexural rigidity of the rigidity variable device 310 increases in regions of the restricted portions 342 compared with when the non-flexure-restricting portions 332 are disposed at the positions corresponding to the restricted portions 342.

A gap is formed between the first longitudinal member 320 and the flexure-restricting portions 334 of the second longitudinal member 330. In this case, when a degree of bending of the restricted portion 342 becomes equal to or higher than a restriction occurrence point, being a specific degree of bending, in the high rigidity state, the flexure-restricting portion 334 restricts an increase in bending of the restricted portion 342, thus increasing flexural rigidity of the rigidity variable device 310 at a portion of the restricted portion 342. As a result, low flexural rigidity of the rigidity variable device 310 is maintained at beginning of bending. However, when the degree of bending reaches or passes a certain value, thus eliminating the gap, rigidity increases rapidly.

As described above, by moving the second longitudinal member 330 relative to the first longitudinal member 320, it is possible to switch rigidity of the rigidity variable device 310 between high rigidity and low rigidity, that is, between a rigid state and a flexible state.

In the low rigidity state, the first longitudinal member 320 easily bends at the low flexural rigidity portions 324. In contrast, in the high rigidity state, the first longitudinal member 320 does not easily bend at the low flexural rigidity portions 324 either. Accordingly, it is possible to say that the rigidity variable device 310 is switched between the low rigidity state and the high rigidity state by an action of locking or unlocking joints.

### Configuration Example 3 of Rigidity Control System

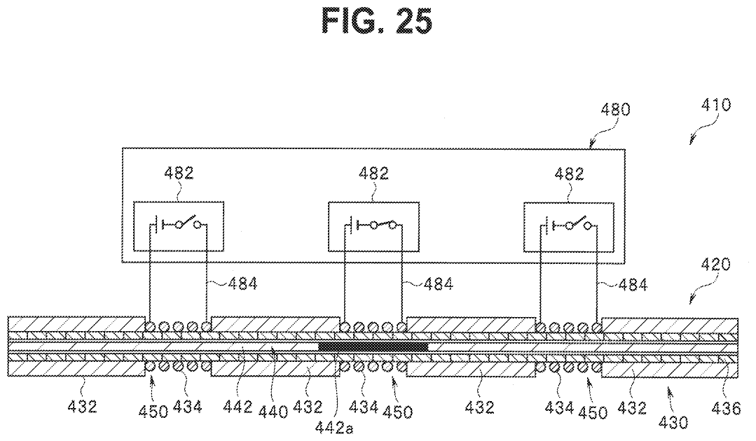

FIG. 25 shows a rigidity control system 410 which is another configuration example applicable for the rigidity variable unit 112. As shown in FIG. 25, the rigidity control system 410 includes a rigidity variable device 420 and a control device 480. The rigidity variable device 420 is mounted on the flexible tube portion 11C. The control device 480 controls the rigidity variable device 420. A portion of a shape memory member 442 in a high rigidity state (rigid state) (heated portion 442a) is shown with black shading.

The rigidity variable device 420 provides different rigidity to the flexible tube portion 11C, thus changing rigidity of the flexible tube portion 11C. The rigidity variable device 420 includes a first longitudinal member 430, a second longitudinal member 440, and inducers 450. The second longitudinal member 440 is disposed along the first longitudinal member 430. For example, the first longitudinal member 430 is an outer sleeve, and the second longitudinal member 440 is a core member disposed in the first longitudinal member 430. For example, the outer sleeve has an annular shape in cross section taken along a line perpendicular to a longitudinal axis of the outer sleeve. An outer periphery of the core member has a circular shape in cross section taken along a line perpendicular to a longitudinal axis of the core member. In this case, the rigidity variable device 420 provides stable flexural rigidity against bending in any direction.

The first longitudinal member 430 includes at least one high flexural rigidity portion 432 and at least one low flexural rigidity portion 434. The high flexural rigidity portion 432 has relatively high flexural rigidity. The low flexural rigidity portion 434 has relatively low flexural rigidity. That is to say, flexural rigidity of the high flexural rigidity portion 432 is high, and flexural rigidity of the low flexural rigidity portion 434 is lower than flexural rigidity of the high flexural rigidity portion 432. The first longitudinal member 430 further includes one cylindrical outer support member 436 that supports the high flexural rigidity portions 432 and the low flexural rigidity portions 434. Flexural rigidity of the outer support member 436 is lower than flexural rigidity of the high flexural rigidity portion 432. Therefore, the first longitudinal member 430 relatively easily bends at the low flexural rigidity portions 434, but does not relatively easily bend at the high flexural rigidity portions 432.

The high flexural rigidity portion 432, the low flexural rigidity portion 434, and the outer support member 436 are separate from each other. The high flexural rigidity portion 432 may be a cylindrical member, such as a metal pipe. The low flexural rigidity portion 434 may be a coil member, such as a coarsely wound coil. The outer support member 436 may be a coil member, such as a closely wound coil. The high flexural rigidity portion 432 is a cylindrical rigid portion having high flexural rigidity. Each of the low flexural rigidity portion 434 and the outer support member 436 is a cylindrical flexible portion having low flexural rigidity.

The outer support member 436 is disposed inside the high flexural rigidity portions 432 and the low flexural rigidity portions 434. An outer peripheral surface of the outer support member 436 is fixed to inner peripheral surfaces of the high flexural rigidity portions 432 by adhesion. The high flexural rigidity portions 432 are arranged at intervals in a direction of a longitudinal axis of the first longitudinal member 430. Each low flexural rigidity portion 434 is disposed in each space formed between the high flexural rigidity portions 432 in the direction of the longitudinal axis of the first longitudinal member 430. Accordingly, the plurality of high flexural rigidity portions 432 and the plurality of low flexural rigidity portions 434 are arranged alternately in the direction of the longitudinal axis of the first longitudinal member 430. End portions of each low flexural rigidity portion 434 are fixed to end portions of the high flexural rigidity portions 432 disposed adjacent to the end portions of the low flexural rigidity portion 434. In each space formed between the high flexural rigidity portions 432, the low flexural rigidity portion 434 is wound around the outer support member 436.

The outer support member 436 extends over an entire length of the rigidity variable device 420. The outer support member 436 is disposed in a spiral shape. For example, the outer support member 436 serves as a core member of the high flexural rigidity portions 432 and the low flexural rigidity portions 434.

The second longitudinal member 440 extends over the entire length of the rigidity variable device 420. The second longitudinal member 440 is disposed in the outer support member 436. An outer peripheral surface of the second longitudinal member 440 is not in contact with an inner peripheral surface of the outer support member 436, and a space is formed between the outer support member 436 and the second longitudinal member 440.

The second longitudinal member 440 includes at least the shape memory member 442. A phase of the shape memory member 442 may shift between a first phase and a second phase due to heat. When the shape memory member 442 is in the first phase, the shape memory member 442 takes a low rigidity state where the shape memory member 442 can easily deform according to an external force, that is, the shape memory member 442 has a low elastic modulus. Accordingly, when the shape memory member 442 is in the first phase, the shape memory member 442 provides relatively low rigidity to the flexible tube portion 11C. When the shape memory member 442 is in the first phase, the rigidity variable device 420 and the flexible tube portion 11C can easily deflect due to an external force, for example.

When the shape memory member 442 is in the second phase, the shape memory member 442 takes the high rigidity state where the shape memory member 442 has higher rigidity than when the shape memory member 442 is in the low rigidity state, that is, the shape memory member 442 has a high elastic modulus. Accordingly, when the shape memory member 442 is in the second phase, the shape memory member 442 takes the high rigidity state where the shape memory member 442 tends to have, against an external force, a memory shape that is memorized in advance. Therefore, the shape memory member 442 provides relatively high rigidity to the flexible tube portion 11C. The memory shape may be a linear shape, for example. When the shape memory member 442 is in the second phase, the rigidity variable device 420 and the flexible tube portion 11C can maintain a substantially linear state, for example, or can slowly deflect due to an external force compared with when the shape memory member 442 is in the first phase.

When the shape memory member 442 is in the first phase, flexural rigidity of the shape memory member 442 is lower than flexural rigidity of the high flexural rigidity portion 432, and is equal to or lower than flexural rigidity of the low flexural rigidity portion 434. When the shape memory member 442 is in the second phase, flexural rigidity of the shape memory member 442 is equal to or lower than flexural rigidity of the high flexural rigidity portion 432, but is higher than flexural rigidity of the low flexural rigidity portion 434.

The low flexural rigidity portion 434 is made of a conductive material. The low flexural rigidity portion 434 may be, a heating wire, for example. That is to say, the low flexural rigidity portion 434 may be a conductive member having large electric resistance. An insulating film not shown in the drawing, for example, is provided around the low flexural rigidity portion 434. The insulating film prevents short circuit between the low flexural rigidity portion 434 and the outer support member 436 and short circuit between the high flexural rigidity portion 432 and the low flexural rigidity portion 434.

An insulating film not shown in the drawing, for example, is provided around the outer support member 436. The insulating film prevents short circuit between the low flexural rigidity portion 434 and the outer support member 436, short circuit between the high flexural rigidity portion 432 and the outer support member 436, and short circuit between the outer support member 436 and the shape memory member 442.

The inducer 450 has the ability to generate heat when supplied with an electric current from the control device 480. The inducer 450 transfers this heat to one portion of the shape memory member 442 disposed around the inducer 450. The inducer 450 then causes phase shift in the shape memory member 442 between the first phase and the second phase at the one portion. The inducer 450 changes rigidity of the one portion of the second longitudinal member 440 in a direction of a longitudinal axis of the second longitudinal member 440.

The control device 480 includes driving units 482 that respectively drive the low flexural rigidity portions 434 independently. Each driving unit 482 includes one power supply and one switch. The driving unit 482 is electrically connected with the low flexural rigidity portion 434 via a wiring portion 484. Each driving unit 482 supplies an electric current to the low flexural rigidity portion 434 via the wiring portion 484 in response to an ON action of the switch. Each driving unit 482 stops supply of an electric current to the low flexural rigidity portion 434 in response to an OFF action of the switch.

The low flexural rigidity portion 434 has the ability to generate heat when supplied with an electric current from the control device 480. A heating value of the low flexural rigidity portion 434 depends on an amount of supply of an electric current. The low flexural rigidity portion 434 serves as the inducer 450 that causes phase shift in the shape memory member 442 between the first phase and the second phase due to heat. To be more specific, the low flexural rigidity portion 434 serves as a coil heater being a heating unit that heats the shape memory member 442 via the outer support member 436. The shape memory member 442 has the property of a phase of the shape memory member 442 shifting from the first phase to the second phase due to heat generated from the low flexural rigidity portion 434 serving as the inducer 450.

When the rigidity control system 410 is in an initial state, the driving units 482 do not supply an electric current to the low flexural rigidity portions 434, so that the low flexural rigidity portions 434 do not generate heat. Therefore, the shape memory member 442 and the flexible tube portion 11C are in a low rigidity state over an entire length.

Each driving unit 482 supplies an electric current to the low flexural rigidity portion 434 via the wiring portion 484 in response to an ON action of the switch. The low flexural rigidity portion 434 generates heat in response to supply of an electric current. Heat is indirectly transferred to the shape memory member 442 from the low flexural rigidity portion 434. Due to transfer of heat, a temperature of the heated portion 442a of the shape memory member 442 increases. Due to heating, a phase of the heated portion 442a switches from the first phase to the second phase, so that the heated portion 442a switches from a low rigidity state to a high rigidity state. With such switching, the flexible tube portion 11C partially switches from a low rigidity state to a high rigidity state. A portion of the flexible tube portion 11C in the high rigidity state maintains a substantially linear state against an external force acting on the flexible tube portion 11C, that is, against a force that can deform the shape memory member 442.

The driving unit 482 stops supply of an electric current to the low flexural rigidity portion 434 in response to an OFF action of the switch. With such an operation, the temperature of the heated portion 442a reduces due to natural cooling. Therefore, the phase of the heated portion 442a switches from the second phase to the first phase, so that rigidity of the heated portion 442a decreases. Rigidity of the flexible tube portion 11C at a portion where the heated portion 442a is located also decreases. Accordingly, the flexible tube portion 11C can easily deflect due to an external force.

As described above, for example, when a phase of a portion of the shape memory member 442 is switched between the first phase and the second phase by the low flexural rigidity portion 434, rigidity of a portion of the flexible tube portion 11C is switched.

### Configuration Example 4 of Rigidity Control System

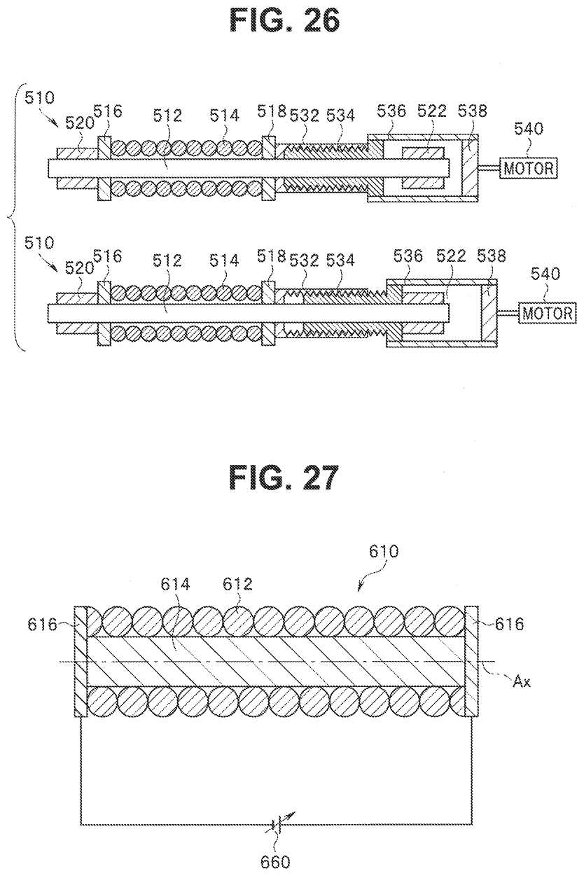

FIG. 26 shows a basic configuration of a rigidity variable device 510 which is another configuration example applicable for the rigidity variable unit 112. The rigidity variable device 510 in a low flexural rigidity state is shown at the top of FIG. 26. The rigidity variable device 510 in a high flexural rigidity state is shown at the bottom of FIG. 26.

The rigidity variable device 510 includes a coil pipe 514 having flexibility, a core wire 512, and a pair of fixing members 520, 522. An example of the coil pipe 514 is a contact coil. The core wire 512 extends through the coil pipe 514. The pair of fixing members 520, 522 are respectively disposed on both sides of the coil pipe 514 and are fixed to the core wire 512.

A washer 516 is disposed between the coil pipe 514 and the fixing member 520. A washer 518 is disposed between the coil pipe 514 and the fixing member 522. The washers 516, 518 have a function of restricting movement of the coil pipe 514 along the core wire 512. The washers 516, 518 prevent the coil pipe 514 from falling off from the core wire 512, and also prevent the fixing members 520, 522 from cutting into the coil pipe 514.

The rigidity variable device 510 also includes an adjusting mechanism that adjusts a gap formed between the coil pipe 514 and each of the fixing members 520, 522. The adjusting mechanism is a pulling mechanism that pulls at least one of the pair of fixing members 520, 522 in a direction in which the pair of fixing members 520, 522 are away from each other. The pulling mechanism includes a nut 532, a lead screw 534, a cylindrical body 536, a lid 538, and a motor 540. The lead screw 534 threadedly engages with the nut 532. The cylindrical body 536 is fixed to the lead screw 534. The lid 538 is fixed to the cylindrical body 536. The motor 540 causes the lead screw 534 to rotate.

The core wire 512 extends in a state of penetrating through the nut 532 and the lead screw 534. The fixing member 522 is accommodated in the cylindrical body 536. The motor 540 is supported such that rotation of the motor 540 per se is prevented, but movement of the motor 540 in an axial direction is allowed. When the lead screw 534 is rotated with respect to the nut 532 by the motor 540, the lead screw 534 can move along an axis of the core wire 512.

In a state shown at the top of FIG. 26, a gap is present between the lead screw 534 and the fixing member 522. In this state, the core wire 512 can move along the coil pipe 514. In such a state, tensile stress is not applied to the core wire 512 when the coil pipe 514 is bent and hence, flexural rigidity is low. The rigidity variable device 510 in the low flexural rigidity state provides low rigidity to the flexible tube portion 11C on which the rigidity variable device 510 is mounted.

In contrast, in a state shown on the lower side of FIG. 26, no gap is present between the lead screw 534 and the fixing member 522. In this state, the core wire 512 cannot move relative to the coil pipe 514. Further, the lead screw 534 presses the fixing member 522, so that tensile stress is applied to the core wire 512. In such a state, tensile stress is further applied to the core wire 512 when the coil pipe 514 is bent and hence, flexural rigidity is high. The rigidity variable device 510 in the high flexural rigidity state provides high rigidity to the flexible tube portion 11C on which the rigidity variable device 510 is mounted.

### Configuration Example 5 of Rigidity Control System

FIG. 27 schematically shows a rigidity variable device 610 and a rigidity control circuit 660 which constitute another configuration example applicable for the rigidity variable unit 112. As shown in FIG. 27, the rigidity variable device 610 includes a coil pipe 612, a conductive polymer artificial muscle 614, and a pair of electrodes 616. The conductive polymer artificial muscle 614 is sealed in the coil pipe 612. The pair of electrodes 616 are provided on both ends of the coil pipe 612. The rigidity variable device 610 is incorporated in the flexible tube portion 11C such that a center axis Ax of the coil pipe 612 is aligned with or parallel to a center axis of the flexible tube portion 11C.

The electrodes 616 of the rigidity variable device 610 are electrically connected with the rigidity control circuit 660. The rigidity control circuit 660 applies a voltage to the conductive polymer artificial muscle 614 via the electrodes 616. When a voltage is applied to the conductive polymer artificial muscle 614, the conductive polymer artificial muscle 614 attempts to increase a diameter thereof about the center axis Ax of the coil pipe 612. However, an increase in the diameter of the conductive polymer artificial muscle 614 is restricted by the coil pipe 612. Therefore, as a higher value of a voltage is applied to the conductive polymer artificial muscle 614, flexural rigidity of the rigidity variable device 610 increases. That is to say, changing rigidity of the rigidity variable device 610 changes flexural rigidity of the flexible tube portion 11C, in which the rigidity variable device 610 is incorporated.

### Configuration Example 6 of Rigidity Control System

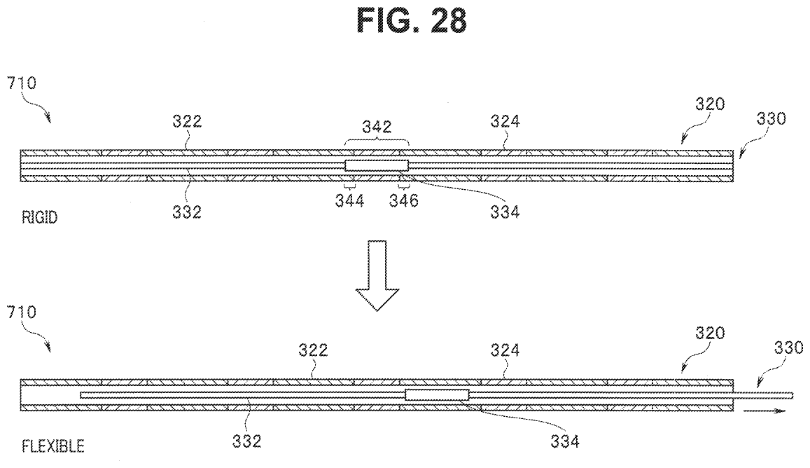

FIG. 28 shows a rigidity variable device 710 which is another configuration example applicable for the rigidity variable unit 112, and FIG. 28 shows switching of rigidity of the rigidity variable device 710 from a high rigidity state to a low rigidity state. The rigidity variable device 710 in the high rigidity state is shown at the top of FIG. 28, and the rigidity variable device 710 in the low rigidity state is shown at the bottom of FIG. 28. In FIG. 28, components substantially equivalent to the corresponding components in FIG. 24 are given the same reference symbols, and the description of such components will be omitted.

As shown in FIG. 28, the second longitudinal member 330 of the rigidity variable device 710 includes a plurality of non-flexure-restricting portions 332 and one flexure-restricting portion 334. Specifically, the second longitudinal member 330 includes two non-flexure-restricting portions 332 and one flexure-restricting portion 334. The non-flexure-restricting portions 332 and the flexure-restricting portion 334 are arranged such that the two non-flexure-restricting portions 332 are disposed on both ends of the one flexure-restricting portion 334 along an axis of the second longitudinal member 330. Other components are substantially equivalent to the corresponding components of the rigidity variable device 310 shown in FIG. 24.

By moving the second longitudinal member 330 relative to the first longitudinal member 320, it is possible to switch rigidity of the rigidity variable device 710 between high rigidity and low rigidity, that is, between a rigid state and a flexible state.

In the rigidity variable device 310 shown in FIG. 24, the number of flexure-restricting portions 334 of the second longitudinal member 330 is equal to the number of low flexural rigidity portions 324. The flexure-restricting portions 334 form core locking portions. The low flexural rigidity portions 324 form joints.

In contrast, in the rigidity variable device 710 of the configuration example 6, the number of flexure-restricting portions 334 of the second longitudinal member 330 is smaller than the number of low flexural rigidity portions 324. The flexure-restricting portions 334 form core locking portions. The low flexural rigidity portions 324 form joints.

In rigidity control that assists endoscopic submucosal dissection (ESD), it is desirable that a position of a portion of the rigidity variable device 710 at which rigidity is increased, that is, a position of a portion that is brought into a linear shape, not be present on a side of distal end portion 11A from the vertex of the “bent portion” formed in the insertion portion 11. This is because, when the portion on the side of the distal end portion 11A from the vertex of the bent portion is brought into a linear shape, a position of the distal end portion 11A of the insertion portion 11 is displaced and hence, performing endoscopic submucosal dissection (ESD) becomes difficult.

In a shape memory alloy method that uses the shape memory member 220 described in the configuration example 1 of the rigidity control system, rigidity of the insertion portion 11 is changed by heating the shape memory member 220. However, in the shape memory alloy method, it takes time for heat of the shape memory member 220 to decrease, that is, it takes time for the insertion portion 11 to be brought into a flexible state. Therefore, fine adjustments for distribution of rigidity in the insertion portion 11 consumes time.

In contrast, the rigidity variable device 710 of the configuration example 6 adopts a joint lock method where joints are locked or unlocked. Further, in the rigidity variable device 710, the number of flexure-restricting portions 334 of the second longitudinal member 330 is smaller than the number of low flexural rigidity portions 324. The flexure-restricting portions 334 form core locking portions. The low flexural rigidity portions 324 form joints. Accordingly, it is possible to promptly perform fine adjustments for a position of a portion of the insertion portion 11 at which rigidity is increased.

The present invention is not limited to the above-mentioned embodiments, and various modifications and applications are conceivable without departing from the gist of the present invention.

What is claimed is:

1. A control device that controls an endoscope having a channel through which a treatment instrument is inserted, the endoscope including an insertion portion configured to be inserted into a subject from a distal end side of the insertion portion and one rigidity variable unit or a plurality of rigidity variable units provided in the insertion portion and configured to be capable of partially changing rigidity of the insertion portion, the control device comprising:
a processor including at least one hardware unit, wherein
the processor detects formation of a bent portion in the insertion portion based on a detection result of a shape of the insertion portion, and
in a case where the processor detects the formation of the bent portion in the insertion portion, the processor performs control of increasing rigidity of the rigidity variable unit located on a proximal end side in the bent portion.

2. The control device according to claim 1, wherein
the processor detects that treatment using the treatment instrument is about to be performed, and
in a case where the processor detects the formation of the bent portion in the insertion portion and detects that the treatment using the treatment instrument is about to be performed, the processor performs the control of increasing the rigidity of the rigidity variable unit located on the proximal end side in the bent portion.

3. The control device according to claim 1, wherein
the processor detects whether the bent portion is pressed against an inner wall surface of a body cavity portion of the subject in a state where the bent portion is formed in the insertion portion, and
in a case where, based on a detection result of whether the bent portion is pressed against the inner wall surface of the body cavity portion of the subject, the processor determines that the bent portion is pressed against the inner wall surface of the body cavity portion of the subject, the processor performs the control of increasing the rigidity of the rigidity variable unit located on the proximal end side in the bent portion.

4. The control device according to claim 1, wherein
the processor determines whether the treatment instrument is inserted through and disposed in the channel, and
in a case where the processor determines, based on a detection result of whether the treatment instrument is inserted through and disposed in the channel, that the treatment instrument is inserted through and disposed in the channel, and in a case where the processor determines, based on a detection result of whether a bent portion is formed in the insertion section, that a predetermined bent portion is formed in the insertion portion, the processor performs the control of increasing the rigidity of the rigidity variable unit located on the proximal end side in the bent portion.

5. The control device according to claim 1, wherein
in a case where the insertion portion is inserted into a body cavity of the subject, the processor calculates stability of the insertion portion, and
in a case where the stability of the insertion portion is less than a predetermined value based on a calculation result of the stability of the insertion portion, the processor continues the control of increasing the rigidity of the rigidity variable unit, and in a case where the stability of the insertion portion is equal to or greater than the predetermined value based on the calculation result of the stability of the insertion portion, the processor stops the control of increasing the rigidity of the rigidity variable unit.

6. The control device according to claim 5, wherein
the processor
determines whether a twisting action is performed while the insertion portion is pressed against an inner wall of a body cavity portion of the subject in a state where the insertion portion is inserted into the body cavity of the subject,
detects relative movement between the insertion portion and the subject from a picked-up image of the subject, and
calculates the stability based on the relative movement between the insertion portion and the subject in a case where the processor determines that the twisting action is performed while the insertion portion is pressed against the inner wall of the body cavity portion of the subject.

7. The control device according to claim 1, wherein
a plurality of the rigidity variable units are arranged in a longitudinal direction of the insertion portion.

8. The control device according to claim 1, wherein
the rigidity variable unit includes a first longitudinal member and a second longitudinal member, the first longitudinal member including a plurality of low flexural rigidity portions, the second longitudinal member being disposed adjacent to and along the first longitudinal member and including a flexure-restricting portion, a number of the flexure-restricting portion being smaller than a number of the plurality of low flexural rigidity portions.

9. The control device according to claim 2, wherein
by receiving a signal based on an operation of the endoscope performed by an operator, the processor detects that the treatment using the treatment instrument is about to be performed.

10. A method for changing rigidity of an insertion portion of an endoscope having a channel through which a treatment instrument is inserted, the endoscope including an insertion portion configured to be inserted into a subject from a distal end side of the insertion portion and one rigidity variable unit or a plurality of rigidity variable units provided in the insertion portion and configured to be capable of partially changing rigidity of the insertion portion,
the method comprising:
detecting formation of a bent portion in the insertion portion; and
increasing rigidity of the rigidity variable unit located on a proximal end side in the bent portion in a case where the formation of the bent portion in the insertion portion is detected.

11. The method for changing rigidity of an insertion portion of an endoscope according to claim 10, wherein
the rigidity variable unit includes a first longitudinal member and a second longitudinal member, the first longitudinal member including a plurality of low flexural rigidity portions, the second longitudinal member being disposed adjacent to and along the first longitudinal member and including a flexure-restricting portion, a number of the flexure-restricting portion being smaller than a number of the plurality of low flexural rigidity portions, and
the rigidity of the rigidity variable unit is switched between a high rigidity state and a low rigidity state by relative movement between the first longitudinal member and the second longitudinal member.

12. A non-transitory recording medium in which a program is recorded, the program causing a computer to execute processing of controlling rigidity of an insertion portion of an endoscope having a channel through which a treatment instrument is inserted, the endoscope including an insertion portion configured to be inserted into a subject from a distal end side of the insertion portion and one rigidity variable unit or a plurality of rigidity variable units provided in the insertion portion and configured to be capable of partially changing the rigidity of the insertion portion, wherein
the non-transitory recording medium causes the computer to execute
processing of detecting formation of a bent portion in the insertion portion based on a detection result of a shape of the insertion portion, and
processing of increasing rigidity of the rigidity variable unit located on a proximal end side in the bent portion in a case where the formation of the bent portion in the insertion portion is detected.
